(19) United States
(12) Patent Application Publication
(10) Pub. No.: US 2024/0216259 A1
(43) Pub. Date: Jul. 4, 2024

(54) ANHYDROUS AGENT FOR DYEING KERATIN MATERIAL, IN PARTICULAR HUMAN HAIR, CONTAINING AMINO SILICONES, PIGMENTS AND PARTICULAR ALKYLENE GLYCOLS

(71) Applicant: Henkel AG & Co. KGaA, Duesseldorf (DE)

(72) Inventors: Constanze KRUCK, Grevenbroich (DE); Sandra HILBIG, Bochum (DE); Melanie MOCH, Dormagen (DE)

(21) Appl. No.: 18/286,344

(22) PCT Filed: Mar. 28, 2022
(86) PCT No.: PCT/EP2022/058059

§ 371 Date: Oct. 10, 2023

(30) Foreign Application Priority Data
Apr. 20, 2021 (DE) 10 2021 203 907.3

(51) Int. Cl. A61K 8/86; A61K 8/34; A61K 8/898

(52) U.S. Cl. CPC A61K 8/86; A61K 8/345; A61K 8/898; A61K 2800/31; A61K 2800/43

(57) ABSTRACT

An anhydrous agent may be used for dyeing keratin material, in particular human hair. The agent may include at least one alkylene glycol of formula (AG),

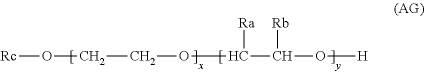

X represents an integer from 0 to 10,000. Y represents an integer from 0 to 10,000. Ra and Rb represent, independently of one another, a hydrogen atom, a C₁-C₆ alkyl group or a hydroxy-C₁-C₆ alkyl group. Rc represents a hydrogen atom, a C₁-C₆ alkyl group, a phenyl group or a benzyl group. And x+y is at least 1. The agent may further include at least one pigment and at least one amino-functionalized silicone polymer.

### CROSS-REFERENCE TO RELATED APPLICATIONS

The present application is a national stage entry according to 35 U.S.C. § 371 of PCT application No.: PCT/EP2022/058059 filed on Mar. 28, 2022; which claims priority to German patent application 10 2021 203 907.3 filed on Apr. 20, 2021; all of which are incorporated herein by reference in their entirety and for all purposes.

### TECHNICAL FIELD

The present application relates to an anhydrous agent for dyeing keratin material, in particular human hair, which contains at least one alkylene glycol of a particular formula (a1), at least one pigment (a2) and at least one amino-functionalized silicone polymer (a3).

This application also relates to a method for dyeing keratin material, in particular human hair, wherein the agent described above is applied to the keratin material and, if appropriate, rinsed out after an exposure time of 30 seconds to 45 minutes.

### BACKGROUND

Changing the shape and color of keratin material, in particular human hair, represents an important field of modern cosmetics. To change the hair color, a person skilled in the art is familiar with a variety of coloring systems depending on the coloring requirements. Oxidation dyes are typically used for permanent, intense dyeing with good fastness properties and good gray coverage. Such coloring agents contain oxidation dye precursors, so-called developer components and coupler components, which together form the actual dyes under the influence of oxidizing agents, such as, for example, hydrogen peroxide. Oxidation dyes are distinguished by very long-lasting color results.

When using direct dyes, already formed dyes diffuse out of the coloring agent into the hair fiber. In comparison with oxidative hair coloring, the colors obtained with direct dyes have a lower durability and a more rapid washing out. Colors with direct dyes usually remain on the hair for a period of between 5 and 20 hair washes.

The use of color pigments for brief changes in color on the hair and/or the skin is known. Color pigments are generally understood to mean insoluble coloring substances. These are present undissolved in the form of small particles in the dyeing formulation and are only deposited from the outside onto the hair fibers and/or the skin surface. They can therefore generally be removed again without residue by washing a few times with surfactant-containing cleaning agents. Various products of this type by the name of hair mascara are available on the market.

If the user desires particularly long-lasting colors, the use of oxidative coloring agents has hitherto been the only option. However, despite multiple optimization attempts, an unpleasant ammonia odor or amine odor cannot be completely avoided in oxidative hair dyeing. The hair damage that remains associated with the use of the oxidative coloring agents also has a disadvantageous effect on the hair of the user. A continuing challenge is therefore the search for alternative, high-performance dyes and dyeing processes. Recently, there has been a particular focus on dyeing systems based on pigments.

### SUMMARY

The object of the present invention is that of providing a coloring agent which makes it possible to fix pigments to the hair in an extremely durable manner. When using the agent in a dyeing method, particularly intensive dyeing results with good wash fastness, good rubbing fastness, good leveling capacity and a particularly uniform color result should be achieved.

Surprisingly, it has been found that the aforementioned object can be achieved in an outstanding manner when keratin material, in particular human hair, is colored with an anhydrous agent which contains at least alkylene glycol of formula (AG) (a1), at least one pigment (a2), and at least one amino-functionalized silicone polymer (a3).

The present invention therefore first relates to an agent for dyeing keratin material, in particular human hair, containing:
(a1) at least alkylene glycol of formula (AG)

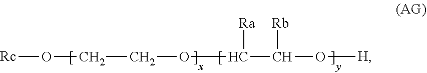

where
x represents an integer from 0 to 10,000,
y represents an integer from 0 to 10,000,
Ra and Rb represent, independently of one another, a hydrogen atom, a C₁-C₆ alkyl group or a hydroxy-C₁-C₆ alkyl group,
Rc represents a hydrogen atom, a C₁-C₆ alkyl group, a phenyl group or a benzyl group, with the proviso that x+y is at least 1, and
(a2) at least one pigment, and
(a3) at least one amino-functionalized silicone polymer.

### DETAILED DESCRIPTION

Within the context of the work leading to this invention, it was discovered that very particularly good color results could be obtained if the pigments or pigments were applied to the core material, in particular human hair, with at least one alkylene glycol of formula (AG) and at least one amino silicones in the form of an anhydrous system. In particular, the wash fastness and the rubbing fastness of the colored hair could be improved.

### Keratin Material

Keratin material is understood to mean hair, skin, and nails (such as, for example, fingernails and/or toenails). Furthermore, wool, furs and feathers also fall under the definition of keratin material.

Keratin material is preferably understood to be human hair, human skin and human nails, in particular fingernails and toenails. Keratin material is very particularly preferably understood to mean the human hair.

### Agent for Dyeing

Within the scope of this invention, the term “agent for dyeing” is used for coloring the keratin material, in particular the hair, brought about by use of pigments. With this dyeing, the pigments as dyeing compounds are deposited in a particularly homogeneous and smooth film on the surface of the keratin material.

According to the invention, the dyeing agent represents a ready-to-use agent. This ready-to-use agent can, for example, be filled into a container and applied to the keratin material in this form without further dilution, mixing or other method steps. For reasons of storage stability, however, it has been found to be very particularly preferred if the ready-to-use cosmetic agent is produced by the hairdresser or user only shortly before use. To prepare the ready-to-use agent, for example the alkylene glycol of formula (AG) (a1) and pigment (a2) can be mixed or predispersed with one or more additional agents, wherein one of these additional agents contains at least one amino-functionalized silicone polymer (a3). However, it is also just as conceivable for the ready-to-use agent to be produced by mixing at least three different agents, wherein one of these agents contains at least one alkylene glycol of formula (AG) (a1), an additional agent contains at least one pigment (a2), and still another agent contains at least amino-functionalized silicone polymer (a3). The means can be mixed, for example, by being shaken and in this way ensures a very particularly uniform distribution of the dispersed pigments. It is characteristic of the ready-to-use agent that it is anhydrous.

### Anhydrous Agent

The agent according to the invention is anhydrous. Within the meaning of the present invention, the term anhydrous is understood to mean that no water is intentionally added to the agent as a separate component. In order to achieve certain desired performance properties, it can be advantageous to add ingredients to the agent which are hygroscopic or contain water as an unavoidable secondary component. In this case, anhydrous means that the agent, based on its total weight, contains less than 1.0 wt. % water, preferably less than 0.5 wt. % water, more preferably less than 0.1 wt. % water, and very particularly preferably less than 0.01 wt. % water. The agent according to the invention explicitly very particularly preferably contains 0 wt. % water.

Expressed otherwise, the present invention therefore first relates to an agent for dyeing keratin material, in particular human hair, containing:
(a1) at least alkylene glycol of formula (AG)

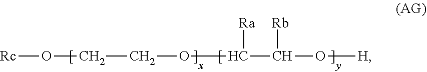

where
x represents an integer from 0 to 10,000,
y represents an integer from 0 to 10,000,
Ra and Rb represent, independently of one another, a hydrogen atom, a C₁-C₆ alkyl group or a hydroxy-C₁-C₆ alkyl group,
Rc represents a hydrogen atom, a C₁-C₆ alkyl group, a phenyl group or a benzyl group, with the proviso that x+y is at least 1, and
(a2) at least one pigment, and
(a3) at least one amino-functionalized silicone polymer,
wherein the agent contains, based on its total weight, less than 1.0 wt. % water, preferably less than 0.5 wt. % water, more preferably less than 0.1 wt. % water, even more preferably less than 0.01 wt. % water and very particularly preferably 0 wt. % water.

### Alkylene Glycols of Formula (AG-I) (a1)

As the second component (a1) essential to the invention, the agent according to the invention contains at least one alkylene glycol of formula (AG),

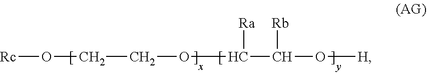

where
x represents an integer from 0 to 10,000,
y represents an integer from 0 to 10,000.
Ra and Rb represent, independently of one another, a hydrogen atom, a C₁-C₆ alkyl group or a hydroxy-C₁-C₆ alkyl group,
Rc represents a hydrogen atom, a C₁-C₆ alkyl group, a phenyl group or a benzyl group, with the proviso that x+y is at least 1, and

Surprisingly, it has been found that the use of at least one specific alkylene glycol of formula (AG) greatly improves the washing resistance of the colorings after applying the agent according to the invention on the keratin material.

The alkylene glycols of formula (AG) are protic substances having at least one hydroxy group. The substituents Ra, Rb and Rc in the compounds of formula (AG-I) are explained below by way of example:

Examples of a C₁-C₆ alkyl group are the groups methyl, ethyl, propyl, isopropyl, n-butyl, s-butyl and t-butyl, n-pentyl and n-hexyl. Propyl, ethyl and methyl are preferred alkyl residues. Preferred examples of a hydroxy-C₁-C₆ alkyl group are a hydroxymethyl, a 2-hydroxyethyl, a 2-hydroxypropyl, a 3-hydroxypropyl, a 4-hydroxybutyl group, a 5-hydroxypentyl and a 6-hydroxyhexyl group; the hydroxymethyl and the 2-hydroxyethyl group are particularly preferred.

By varying the functional groups Ra, Rb and Rc as well as x and y, the polarity of the alkylene glycol (AG) can be adjusted, and the viscosity of the agent according to the invention can be influenced.

In the alkylene glycols (a1) of formula (AG), x can represent an integer from 0 to 10,000.

The functional group y can represent an integer from 0 to 10,000. In this case, the proviso exists that the sum of x and y must represent a value of at least 1. If y represents the number 0, the functional groups Ra and Rb are not present in the alkylene glycols of the formula (AG).

The R_{c} functional group represents a hydrogen atom, a C₁-C₆ alkyl group, a phenyl group or a benzyl group. Within the scope of this embodiment, it is furthermore very particularly preferred if Rc represents a hydrogen atom.

The use of certain alkylene glycols of formula (AG) has proven to be very particularly suitable for producing colorings having good wash fastness and good rub fastness. In the context of this embodiment, the use of polyethylene glycols is therefore very particularly preferred.

Polyethylene glycols are alkylene glycols of formula (AG) in which x represents an integer from 1 to 10,000, in particular an integer from 2 to 10,000, and y represents the number 0. Rc represents a hydrogen atom.

Within the scope of another very particularly preferred embodiment, an agent according to the invention is characterized in that it contains:
(a1) at least alkylene glycol of formula (AG), where
x represents an integer from 1 to 10,000, and
y represents the number 0, and
Rc represents a hydrogen atom.

The corresponding polyethylene glycols are compounds of formula (AG-1):

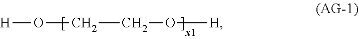

where x1 represents an integer from 1 to 10,000.

In the context of the work leading to this invention, it was found that the polyethylene glycols exhibit particularly favorable suitability for, on the one hand, improving the fastness properties of the coloring agents and for, on the other hand, optimally adjusting the viscosity of the agents.

Depending on their chain length, polyethylene glycols are liquid or solid water-soluble polymers. Polyethylene glycols with a molecular mass between 200 g/mol and 400 g/mol are non-volatile liquids at room temperature. PEG 600 has a melting range of 17 to 22° C. and therefore a pasty consistency. With molecular masses above 3,000 g/mol, the PEGs are solid substances and are commercially available as flakes or powders.

Especially the use of low molecular weight polyethylene glycols has proven to be well-suited for achieving the object of the invention. In the case of low molecular weight polyethylene glycols in the context of the present invention, x1 represents an integer from 1 to 100, preferably an integer from 1 to 80, more preferably an integer from 2 to 60, even more preferably an integer from 3 to 40, even more preferably an integer from 4 to 20, and very particularly preferably an integer from 6 to 15.

Within the scope of another very particularly preferred embodiment, an agent according to the invention is characterized in that it contains:
(a1) at least one alkylene glycol of formula (AG-1), where

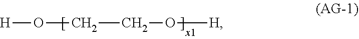

x1 represents an integer from 1 to 100, preferably an integer from 1 to 80, more preferably an integer from 2 to 60, even more preferably an integer from 3 to 40, even more preferably an integer from 4 to 20, and very particularly preferably an integer from 6 to 15.

A very particularly preferred low molecular weight polyethylene glycol is PEG-8, for example. PEG-8 comprises 8 ethylene glycol units (x1=8), has an average molecular weight of 400 g/mol, and bears the CAS number 25322-68-3. PEG-8 is alternatively also referred to as PEG 400 and is commercially available, for example, from APS.

Additional well-suited low molecular weight polyethylene glycols are, for example, PEG-6, PEG-7, PEG-9 and PEG-10.

Another well-suited polyethylene glycol is PEG-32, for example. PEG-32 comprises 32 ethylene glycol units (x1=32), has an average molar mass of 1,500 g/mol and bears the CAS number 25322-68-3. PEG-32 is alternatively also referred to as PEG 1500 and can, for example, be purchased commercially from Clariant.

Furthermore, the use of high molecular weight polyethylene glycols for achieving the object according to the invention has also proven to be well suited.

High molecular weight polyethylene glycols within the meaning of the present invention can be represented by the formula (AG-2), where the index number x2 represents an integer from 101 to 10,000

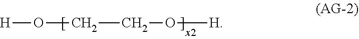

In the case of very well-suited high molecular weight polyethylene glycols, x2 represents an integer from 101 to 1,000, preferably an integer from 105 to 800, more preferably an integer from 107 to 600, even more preferably an integer from 109 to 400, and very particularly preferably an integer from 110 to 200.

Within the scope of another very particularly preferred embodiment, an agent according to the invention is characterized in that it contains:
(a1) at least one alkylene glycol of formula (AG-2), where

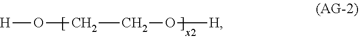

x2 represents an integer from 101 to 1,000, preferably an integer from 105 to 800, more preferably an integer from 107 to 600, even more preferably an integer from 109 to 400, and very particularly preferably an integer from 110 to 200.

A particularly well-suited high molecular weight polyethylene glycol is, for example, PEG 6000, which can be obtained commercially from National Starch (China). The molecular weight of PEG 6000 is 6,000 to 7,500 g/mol, corresponding to an x2 value of 136 to 171.

Another well-suited polyethylene glycol is PEG 12000 which, for example, is commercially sold by CG chemicals under the trade name of polyethylene glycol 12000 S (or PEG 12000 S). The molecular weight of PEG 12000 is given at 10,500 to 15,000 g/mol, corresponding to an x2 value of 238 to 341.

Another well-suited polyethylene glycol is also PEG 20000 which is commercially available under the trade name polyglycol 20000 P or under the alternative name PEG-350 from Clariant. For PEG 20000, an average molecular weight of 20,000 g/mol is given which corresponds to an x2 value of 454.

Surprisingly, it has been found that coloring agents which contain both a low molecular weight alkylene glycol (AG-1) and a high molecular weight alkylene glycol (AG-2) have particularly favorable performance properties since these agents have very good fastness properties and are also optimized with regard to their rheological profile.

Within the scope of another very particularly preferred embodiment, an agent according to the invention is characterized in that it contains:
(a11) at least one first alkylene glycol of formula (AG-1), where

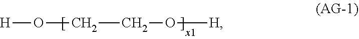

x1 represents an integer from 1 to 100, preferably an integer from 1 to 80, more preferably an integer from 2 to 60, even more preferably an integer from 3 to 40, even more preferably an integer from 4 to 20, and very particularly preferably an integer from 6 to 15, and
(a12) at least one second alkylene glycol of formula (AG-2), where

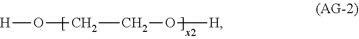

x2 represents an integer from 101 to 1,000, preferably an integer from 105 to 800, more preferably an integer from 107 to 600, even more preferably an integer from 109 to 400, and very particularly preferably an integer from 110 to 200.

Within the scope of another embodiment, the agent according to the invention can also contain at least one alkylene glycol of formula (AG-3),

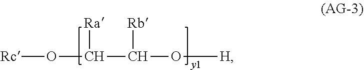

where
y1 represents an integer from 1 to 1,000, preferably an integer from 3 to 100, particularly preferably an integer from 5 to 50,
Ra′ and Rb′ represent, independently of one another, a hydrogen atom or a C₁-C₆ alkyl group, wherein preferably one of the functional groups of Ra′ and Rb′ is a hydrogen atom, and the remaining of the two functional groups is a C₁-C₆ alkyl group, preferably a methyl group, and
Rc′ represents a C₁-C₆ alkyl group.

Within the scope of another preferred embodiment, an agent according to the invention is therefore characterized in that it contains:
(a1) at least alkylene glycol of formula (AG-3),

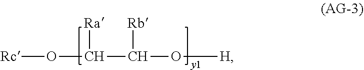

where
y1 represents an integer from 1 to 1,000, preferably an integer from 3 to 100, particularly preferably an integer from 5 to 50,
Ra′ and Rb′ represent, independently of one another, a hydrogen atom or a C₁-C₆ alkyl group, wherein preferably one of the functional groups of Ra′ and Rb′ is a hydrogen atom, and the remaining of the two functional groups is a C₁-C₆ alkyl group, preferably a methyl group, and
Rc′ represents a C₁-C₆ alkyl group.

A very particularly suitable alkylene glycol of formula (AG-3) is propylene glycol monobutyl ether which is also referred to as PPG-14 butyl ether and bears the CAS number 9003-13-8. PPG-14 butyl ether can be purchased commercially under the trade name Ucon Fluid AP from Dow.

Within the scope of another very particularly preferred embodiment, an agent according to the invention is therefore also characterized in that it contains:
(a1) PPG-14 butyl ether.

The alkylene glycol of formula (AG-3) can be used either as the sole alkylene glycol or else in combination with additional alkylene glycols, in particular the alkylene glycols of formula (AG-1) and/or (AG-2).

Very positive effects can therefore be obtained with an agent according to the invention which is characterized in that contains:
(a11) at least one alkylene glycol of formula (AG-1), where

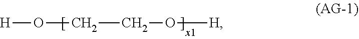

x1 represents an integer from 1 to 100, preferably an integer from 1 to 80, more preferably an integer from 2 to 60, even more preferably an integer from 3 to 40, even more preferably an integer from 4 to 20, and very particularly preferably an integer from 6 to 15, and
(a13) at least alkylene glycol of formula (AG-3),

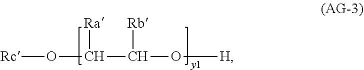

where
y1 represents an integer from 1 to 1,000, preferably an integer from 3 to 100, particularly preferably an integer from 5 to 50,
Ra′ and Rb′ represent, independently of one another, a hydrogen atom or a C₁-C₆ alkyl group, wherein preferably one of the functional groups of Ra′ and Rb′ is a hydrogen atom, and the remaining of the two functional groups is a C₁-C₆ alkyl group, preferably a methyl group, and
Rc′ represents a C₁-C₆ alkyl group.

Positive effects can also be obtained with an agent according to the invention which is
characterized in that it contains:
(a12) at least one alkylene glycol of formula (AG-2), where

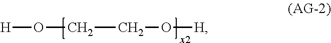

x2 represents an integer from 101 to 1,000, preferably an integer from 105 to 800, more preferably an integer from 107 to 600, even more preferably an integer from 109 to 400, and very particularly preferably an integer from 110 to 200, and
(a13) contains at least alkylene glycol of formula (AG-3),

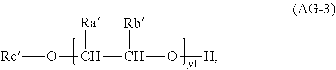

where
y1 represents an integer from 1 to 1,000, preferably an integer from 3 to 100, particularly preferably an integer from 5 to 50,
Ra′ and Rb′ represent, independently of one another, a hydrogen atom or a C₁-C₆ alkyl group, wherein preferably one of the functional groups of Ra′ and Rb′ is a hydrogen atom, and the remaining of the two functional groups is a C₁-C₆ alkyl group, preferably a methyl group, and
Rc′ represents a C₁-C₆ alkyl group.

Very positive effects can also be obtained with an agent according to the invention which is characterized in that it contains:
(a11) at least one alkylene glycol of formula (AG-1), where

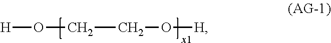

x1 represents an integer from 1 to 100, preferably an integer from 1 to 80, more preferably an integer from 2 to 60, even more preferably an integer from 3 to 40, even more preferably an integer from 4 to 20, and very particularly preferably an integer from 6 to 15, and
(a12) at least one alkylene glycol of formula (AG-2), where

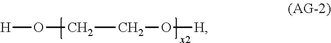

x2 represents an integer from 101 to 1,000, preferably an integer from 105 to 800, more preferably an integer from 107 to 600, even more preferably an integer from 109 to 400, and very particularly preferably an integer from 110 to 200, and
(a13) at least alkylene glycol of formula (AG-3),

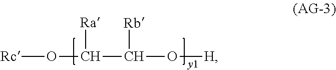

where
y1 represents an integer from 1 to 1,000, preferably an integer from 3 to 100, particularly preferably an integer from 5 to 50,
Ra′ and Rb′ represent, independently of one another, a hydrogen atom or a C₁-C₆ alkyl group, wherein preferably one of the functional groups of Ra′ and Rb′ is a hydrogen atom, and the remaining of the two functional groups is a C₁-C₆ alkyl group, preferably a methyl group, and
Rc′ represents a C₁-C₆ alkyl group.

Since the agent according to the invention is anhydrous, it is also necessary to select the cosmetic carrier of the components other than water. In this context, it has been found to be very particularly preferred if the alkylene glycols or the alkylene glycols (AG), and in particular the particularly preferred compounds of formula (AG-1), (AG-2) and/or (AG-3), themselves act as cosmetic carrier substances. For this purpose, the alkylene glycols (AG) are particularly preferably used in the correspondingly high quantity ranges in the agent according to the invention. These amounts of use can be, for example, based on the total weight of the agent, in a range from 10.0 to 99.0 wt. %, preferably from 30.0 to 99.0 wt. %, more preferably from 50.0 to 99.0 wt. %, and very particularly preferably from 70.0 to 99.0 wt. %.

Within the scope of another very particularly preferred embodiment, an agent according to the invention is characterized in that it contains, based on the total weight of the agent, one or more alkylene glycols corresponding to formula (AG) (a1) in a total amount from 10.0 to 99.0 wt. %, preferably from 30.0 to 99.0 wt. %, more preferably from 50.0 to 99.0 wt. %, and very particularly preferably from 70.0 to 99.0 wt. %.

The agent according to the invention preferably contains, based on the total weight of the agent, (a11), one or more alkylene glycols of formula (AG-1) in a total amount from 20.0 to 99.0 wt. %, preferably from 40.0 to 95.0 wt. %, particularly preferably from 60.0 to 90.0 wt. %.

The agent according to the invention preferably contains, based on the total weight of the agent, (a12) one or more alkylene glycols of formula (AG-2) in a total amount from 1.0 to 35.0 wt. %, preferably from 3.0 to 30.0 wt. %, particularly preferably from 4.0 to 25.0 wt. %.

The agent according to the invention contains, based on the total weight of the agent, (a13) one or more alkylene glycols of formula (AG-3) in a total amount from 0.1 to 20.0 wt. %, preferably from 0.2 to 10.0 wt. %, still more preferably from 0.4 to 5.0 wt. %, still more preferably from 0.5 to 3.0 wt. %, and very particularly preferably from 0.5 to 1.5 wt. %.

Within the scope of another very particularly preferred embodiment, an agent according to the invention is characterized in that it contains, based on the total weight of the agent, (a1) one or more alkylene glycols of formula (AG-1) in a total amount from 20.0 to 99.0 wt. %, preferably from 40.0 to 95.0 wt. %, more preferably from 60.0 to 90.0 wt. %, and/or contains one or more alkylene glycols of formula (AG-2) in a total amount from 1.0 to 35.0 wt. %, preferably from 3.0 to 30.0 wt. %, particularly preferably from 4.0 to 25.0 wt. %, and/or contains one or more alkylene glycols of formula (AG-3) in a total amount from 0.1 to 20.0 wt. %, preferably from 0.2 to 10.0 wt. %, particularly preferably from 0.4 to 5.0 wt. %.

Within the scope of another very particularly preferred embodiment, an agent according to the invention is characterized in that it contains, based on the total weight of the agent, (a11) one or more alkylene glycols of formula (AG-1) in a total amount from 20.0 to 99.0 wt. %, and
(a12) one or more alkylene glycols of formula (AG-2) in a total amount from 1.0 to 35.0 wt. %.

Within the scope of another very particularly preferred embodiment, an agent according to the invention is characterized in that it contains, based on the total weight of the agent, (a11) one or more alkylene glycols of formula (AG-1) in a total amount from 40.0 to 95.0 wt. %, and
(a12) one or more alkylene glycols of formula (AG-2) in a total amount from 3.0 to 30.0 wt. %.

Within the scope of another very particularly preferred embodiment, an agent according to the invention is characterized in that it contains, based on the total weight of the agent, (a11) one or more alkylene glycols of formula (AG-1) in a total amount from 60.0 to 90.0 wt. %, and
(a12) one or more alkylene glycols of formula (AG-2) in a total amount from 4.0 to 25.0 wt. %.

Within the scope of another very particularly preferred embodiment, an agent according to the invention is characterized in that it contains, based on the total weight of the agent, (a11) one or more alkylene glycols of formula (AG-1) in a total amount from 20.0 to 99.0 wt. %, and
(a12) one or more alkylene glycols of formula (AG-2) in a total amount from 1.0 to 35.0 wt. %, and
(a13) one or more alkylene glycols of formula (AG-3) in a total amount from 0.1 to 20.0 wt. %.

Within the scope of another very particularly preferred embodiment, an agent according to the invention is characterized in that it contains, based on the total weight of the agent, (a11) one or more alkylene glycols of formula (AG-1) in a total amount from 40.0 to 95.0 wt. %, and
(a12) one or more alkylene glycols of formula (AG-2) in a total amount from 3.0 to 30.0 wt. %,
(a13) one or more alkylene glycols of formula (AG-3) in a total amount from 0.2 to 10.0 wt. %.

Within the scope of another very particularly preferred embodiment, an agent according to the invention is characterized in that it contains, based on the total weight of the agent, (a11) one or more alkylene glycols of formula (AG-1) in a total amount from 60.0 to 90.0 wt. %, and
(a12) one or more alkylene glycols of formula (AG-2) in a total amount from 4.0 to 25.0 wt. %, and
(a13) one or more alkylene glycols of formula (AG-3) in a total amount of 0.4 to 5.0 wt. %.

It is understood here that the sum of (a1) all of the alkylene glycols (AG), pigments (a2) and amino-functionalized silicone polymers (a3) present in the agents cannot be any more than 100 wt. %. If still other optional ingredients are contained in the agent, the total sum of (a1), (a2) and (a3) decreases to a corresponding extent to values less than 100 wt. %.

### Pigments (a2)

As the second essential component, the agent according to the invention contains at least one pigment (a2). Pigments within the meaning of the present invention are understood to mean coloring compounds which have a solubility of less than 0.5 g/L, preferably of less than 0.1 g/L, even more preferably of less than 0.05 g/L, at 25° C. in water. The method described below, for example, can be used to determine water solubility: 0.5 g of the pigment is weighed out in a beaker. A stir bar is added. Then one liter of distilled water is added. This mixture is heated to 25° C. while stirring with a magnetic stirrer for one hour. If still undissolved components of the pigment are visible in the mixture after this period, the solubility of the pigment is below 0.5 g/L. If the pigment-water mixture cannot be visually assessed due to the high intensity of the pigment that may be finely dispersed, the mixture is filtered. If a portion of undissolved pigments remains on the filter paper, the solubility of the pigment is below 0.5 g/L.

Suitable color pigments can be of inorganic and/or organic origin.

In a preferred embodiment, an agent according to the invention is characterized in that it contains at least one coloring compound (a2) from the group consisting of inorganic and/or organic pigments.

Preferred color pigments are selected from synthetic or natural inorganic pigments. Inorganic color pigments of natural origin can be produced, for example, from chalk, ocher, umbra, green soil, burnt Siena or graphite. Furthermore, black pigments such as, for example, iron oxide black, chromatic pigments such as, for example, ultramarine or iron oxide red, and also fluorescent or phosphorescent pigments, can be used as inorganic color pigments.

Colored metal oxides, hydroxides and oxide hydrates, mixed phase pigments, sulfur-containing silicates, silicates, metal sulfides, complex metal cyanides, metal sulfates, chromates and/or molybdates are particularly suitable. Particularly preferred color pigments are black iron oxide (CI 77799), yellow iron oxide (CI 777492), red and brown iron oxide (CI 777491), manganese violet (CI 77742), ultramarine (sodium aluminum sulphosilicates, CI 77007, Pigment Blue 29), chromium oxide hydrate (CI 77289), Iron Blue ferric ferrocyanide, CI 77510) and/or carmine (cochineal).

Color pigments which are likewise particularly preferred according to the invention are colored pearlescent pigments. These are usually based on mica and may be coated with one or more metal oxides. Mica is a phyllosilicate. The most important representatives of these silicates are muscovite, phlogopite, paragonite, biotite, lepidolite, and margarite. In order to produce the pearlescing pigments in conjunction with metal oxides, mica, primarily muscovite or phlogopite, is coated with a metal oxide.

As an alternative to natural mica, synthetic mica coated with one or more metal oxides(s) can also be used as a pearlescent pigment. Particularly preferred pearlescent pigments are based on natural or synthetic mica and are coated with one or more of the aforementioned metal oxides. The color of the respective pigments can be varied by varying the layer thickness of the metal oxide(s).

In another preferred embodiment, an agent according to the invention is characterized in that it contains at least one inorganic pigment (a2) preferably selected from the group consisting of colored metal oxides, metal hydroxides, metal oxide hydrates, silicates, metal sulfides, complex metal cyanides, metal sulphates, bronze pigments and/or from mica-based colored pigments which are coated with at least one metal oxide and/or a metal oxychloride.

In another preferred embodiment, an agent according to the invention is characterized in that it contains at least one coloring compound (a2) from the group consisting of pigments selected from mica-based colored pigments which are coated with one or more metal oxides from the group consisting of titanium dioxide (CI 77891), black iron oxide (CI 77499), yellow iron oxide (CI 77492), red and/or brown iron oxide (CI 77491, CI 77499), manganese violet (CI 77742), ultramarine (sodium aluminum sulfosilicates, CI 77007, pigment blue 29), chromium oxide hydrate (CI 77289), chromium oxide (CI 77288) and/or iron blue (ferric ferrocyanide, CI 77510).

Examples of particularly suitable color pigments are commercially available, for example, under the trade names Rona®, Colorona®, Xirona®, Dichrona® and Timiron® from the company Merck, Ariabel® and Unipure® from the company Sensient, Prestige® from the company Eckart Cosmetic Colors, and Sunshine® from the company Sunstar.

Very particularly preferred color pigments with the trade name Colorona® are, for example:
Colorona Copper, Merck, MICA, CI 77491 (IRON OXIDES)
Colorona Passion Orange, Merck, Mica, CI 77491 (IRON OXIDES), Alumina
Colorona Patina Silver, Merck, MICA, CI 77499 (IRON OXIDES), CI 77891 (TITANIUM DIOXIDE)
Colorona RY, Merck, CI 77891 (TITANIUM DIOXIDE), MICA, CI 75470 (CARMINE)
Colorona Oriental Beige, Merck, MICA, CI 77891 (TITANIUM DIOXIDE), CI 77491 (IRON OXIDES)
Colorona Dark Blue, Merck, MICA, TITANIUM DIOXIDE, FERRIC FERROCYANIDE
Colorona Chameleon, Merck, CI 77491 (IRON OXIDES), MICA
Colorona Aborigine Amber, Merck, MICA, CI77499 (IRON OXIDES), CI77891 (TITANIUM DIOXIDE)
Colorona Blackstar Blue, Merck, CI 77499 (IRON OXIDES), MICA
Colorona Patagonian Purple, Merck, MICA, CI 77491 (IRON OXIDES), CI 77891 (TITANIUM DIOXIDE), CI 77510 (FERRIC FERROCYANIDE)
Colorona Red Brown, Merck, MICA, CI 77491 (IRON OXIDES), CI 77891 (TITANIUM DIOXIDE)
Colorona Russet, Merck, CI 77491 (TITANIUM DIOXIDE), MICA, CI 77891 (IRON OXIDES)
Colorona Imperial Red, Merck, MICA, TITANIUM DIOXIDE (CI 77891), D&C RED NO. 30 (CI 73360)
Colorona Majestic Green, Merck, CI 77891 (TITANIUM DIOXIDE), MICA, CI 77288 (CHROMIUM OXIDE GREENS)
Colorona Light Blue, Merck, MICA, TITANIUM DIOXIDE (CI 77891), FERRIC FERROCYANIDE (CI 77510)
Colorona Red Gold, Merck, MICA, CI 77891 (TITANIUM DIOXIDE), CI 77491 (IRON OXIDES)
Colorona Gold Plus MP 25, Merck, MICA, TITANIUM DIOXIDE (CI77891), IRON OXIDES (CI 77491)
Colorona Carmine Red, Merck, MICA, TITANIUM DIOXIDE, CARMINE
Colorona Blackstar Green, Merck, MICA, CI 77499 (IRON OXIDES)
Colorona Bordeaux, Merck, MICA, CI 77491 (IRON OXIDES)
Colorona Bronze, Merck, MICA, CI 77491 (IRON OXIDES)
Colorona Bronze Fine, Merck, MICA, CI 77491 (IRON OXIDES)
Colorona Fine Gold MP 20, Merck, MICA, CI 77891 (TITANIUM DIOXIDE), CI 77491 (IRON OXIDES)
Colorona Sienna Fine, Merck, CI 77491 (IRON OXIDES), MICA
Colorona Sienna, Merck, MICA, CI 77491 (IRON OXIDES)
Colorona Precious Gold, Merck, Mica, CI 77891 (Titanium dioxide), Silica, CI 77491 (Iron Oxides), Tin oxide
Colorona Sun Gold Sparkle MP 29, Merck, MICA, TITANIUM DIOXIDE, IRON OXIDES, MICA, CI 77891, CI 77491 (EU)
Colorona Mica Black, Merck, CI 77499 (Iron oxides), Mica, CI 77891 (Titanium dioxide)
Colorona Bright Gold, Merck, Mica, CI 77891 (Titanium dioxide), CI 77491 (Iron oxides)
Colorona Blackstar Gold, Merck, MICA, CI 77499 (IRON OXIDES)
Additional particularly preferred color pigments with the trade name Xirona® are, for example:
Xirona Golden Sky, Merck, Silica, CI 77891 (Titanium Dioxide), Tin Oxide
Xirona Caribbean Blue, Merck, Mica, CI 77891 (Titanium Dioxide), Silica, Tin Oxide
Xirona Kiwi Rose, Merck, Silica, CI 77891 (Titanium Dioxide), Tin Oxide
Xirona Magic Mauve, Merck, Silica, CI 77891 (Titanium Dioxide), Tin Oxide.
In addition, particularly preferred color pigments with the trade name Unipure® are, for example:
Unipure Red LC 381 EM, Sensient CI 77491 (Iron Oxides), Silica
Unipure Black LC 989 EM, Sensient, CI 77499 (Iron Oxides), Silica
Unipure Yellow LC 182 EM, Sensient, CI 77492 (Iron Oxides), Silica

In another embodiment, the agent according to the invention can also contain (a2) one or more coloring compounds from the group consisting of organic pigments.

The organic pigments according to the invention are correspondingly insoluble organic dyes or color lakes which may be selected, for example, from the group of nitroso, nitro, azo, xanthene, anthraquinone, isoindolinone, isoindoline, quinacridone, perinone, perylene, diketopyrrolopyorrole, indigo, thioindido, dioxazine, and/or triarylmethane compounds.

Particularly well suited organic pigments can for example include carmine, quinacridone, phthalocyanine, sorghum, blue pigments with the Color Index numbers CI 42090, CI 69800, CI 69825, CI 73000, CI 74100 or CI 74160, yellow pigments with the Color Index numbers CI 11680, CI 11710, CI 15985, CI 19140, CI 20040, CI 21100, CI 21108, CI 47000 or CI 47005, green pigments with the Color Index numbers CI 61565, CI 61570 or CI 74260, orange pigments with the Color Index numbers CI 11725, CI 15510, CI 45370 or CI 71105, and red pigments with the Color Index numbers CI 12085, CI 12120, CI 12370, CI 12420, CI 12490, CI 14700, CI 15525, CI 15580, CI 15620, CI 15630, CI 15800, CI 15850, CI15865, CI15880, CI17200, CI26100, CI45380, CI45410, CI58000, CI 73360, CI 73915 and/or CI 75470.

In another particularly preferred embodiment, an agent according to the invention is characterized in that it contains at least one organic pigment (a2) preferably selected from the group consisting of carmine, quinacridone, phthalocyanine, sorghum, blue pigments with the Color Index numbers CI 42090, CI 69800, CI 69825, CI 73000, CI 74100 or CI 74160, yellow pigments with the Color Index numbers CI 11680, CI11710, CI15985, CI19140, CI20040, CI21100, CI21108, CI47000 or CI 47005, green pigments with the Color Index numbers CI 61565, CI 61570 or CI 74260, orange pigments with the Color Index numbers CI 11725, CI 15510, CI 45370 or CI 71105, and red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

The organic pigment can also be a color lake. The term color lake within the scope of the invention is understood to mean particles which comprise a layer of absorbed dyes, wherein the unit consisting of particles and dye is insoluble under the above mentioned conditions. The particles may be, for example, inorganic substrates which may be aluminum, silica, calcium borosilicate, calcium aluminum borosilicate or aluminum.

For example, the alizarin color lake can be used as the color lake.

Owing to their excellent light and temperature resistance, the use of the aforementioned pigments in the method according to the invention is very particularly preferred. It is further preferred if the pigments used have a certain particle size. It is therefore advantageous according to the invention if the at least one pigment has an average particle size D₅₀ from 1.0 to 50 μm, preferably from 5.0 to 45 μm, preferably from 10 to 40 μm, in particular from 14 to 30 μm. The average particle size D₅₀ can be determined, for example, using dynamic light scattering (DLS).

The pigments (a2) represent the second essential of the agent according to the invention and are preferably used within certain quantity ranges in the agent.

Particularly positive results were obtained when the agent contained, based on the total weight of the agent, one or more pigments (a2) in a total amount from 0.01 to 10.0 wt. %, preferably 0.1 to 5.0 wt. %, more preferably from 0.2 to 2.5 wt. %, and very particularly preferably from 0.25 to 1.5 wt. %.

In another very particularly preferred embodiment, an agent according to the invention is characterized in that the agent contains, based on the total weight of the agent, one or more pigments (a2) in a total amount from 0.01 to 10.0 wt. %, preferably 0.1 to 5.0 wt. %, more preferably from 0.2 to 2.5 wt. %, and very particularly preferably from 0.25 to 1.5 wt. %.

In another very particularly preferred embodiment, an agent according to the invention is characterized in that the agent contains, based on the total weight of the agent, one or more inorganic pigments (a2) in a total amount from 0.01 to 10.0 wt. %, preferably 0.1 to 5.0 wt. %, more preferably from 0.2 to 2.5 wt. %, and very particularly preferably from 0.25 to 1.5 wt. %.

In another very particularly preferred embodiment, an agent according to the invention is characterized in that the agent contains, based on the total weight of the agent, one or more organic pigments (a2) in a total amount from 0.01 to 10.0 wt. %, preferably 0.1 to 5.0 wt. %, more preferably from 0.2 to 2.5 wt. %, and very particularly preferably from 0.25 to 1.5 wt. %.

Amino-functionalized silicone polymers (a3)

As a third ingredient essential to the invention, the agent according to the invention contains at least one amino-functionalized silicone polymer (a3). The amino-functionalized silicone polymer can alternatively also be referred to as amino silicones or amodimethicone.

Silicone polymers are generally macromolecules with a molecular weight of at least 500 g/mol, preferably at least 1,000 g/mol, more preferably at least 2,500 g/mol and more preferably at least 5,000 g/mol which comprise repeating organic units.

The maximum molecular weight of the silicone polymer depends on the degree of polymerization (number of polymerized monomers), and the batch size and is also determined by the polymerization method. In the context of the present invention, it is preferable that the maximum molecular weight of the silicone polymers is not more than 10⁷ g/mol, preferably not more than 106 g/mol, and particularly preferably not more than 10⁵ g/mol.

The silicone polymers comprise many Si—O repeat units, wherein the Si atoms can bear organic residues such as, for example, alkyl groups or substituted alkyl groups. Alternatively, a silicone polymer is therefore also referred to as polydimethylsiloxane.

Corresponding to the high molecular weight of the silicone polymers, these are based on more than 10 Si—O repeat units, preferably more than 50 Si—O repeat units and particularly preferably more than 100 Si—O repeat units, very particularly preferably more than 500 Si—O repeat units.

An amino-functionalized silicone polymer is understood to mean a functionalized silicone which carries at least one structural unit with an amino group. The amino-functionalized silicone polymer preferably carries a plurality of structural units with at least one amino group in each case. An amino group is understood to mean a primary amino group, a secondary amino group and a tertiary amino group. All these amino groups can be protonated in an acidic environment and are then present in their cationic form.

In principle, it was possible to achieve positive effects of amino-functionalized silicone polymers (a3) when they bore at least one primary, at least one secondary and/or at least one tertiary amino group. However, colorings with the highest color intensities were observed when an amino-functionalized silicone polymer (a3) was used in an agent containing at least one secondary amino group.

In a very particularly preferred embodiment, an agent according to the invention is characterized in that it also contains:
(a3) at least one amino-functionalized silicone polymer having at least one secondary amino group.

The secondary amino groups(s) can be at different positions in the amino-functionalized silicone polymer. A particularly good effect was found when an amino-functionalized silicone polymer (a3) was used that had at least one, preferably a plurality of, structural units of the formula (Si-amino).

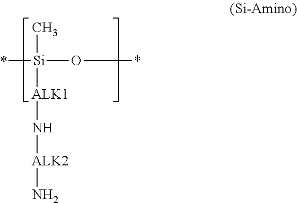

In the structural units of formula (Si-amino), the abbreviations ALK1 and ALK2 are each independently a linear or branched divalent C₁-C₂₀ alkylene group.

In an additional very particularly preferred embodiment, an agent according to the invention is characterized In that the agent contains at least one amino-functionalized silicone polymer (a3) which comprises at least one structural unit of formula (Si-amino),

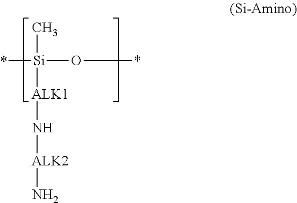

where
ALK1 and ALK2 represent, independently of one another, a linear or branched divalent C₁-C₂₀ alkylene group.

The position marked with an asterisk (*) always indicates the bond to other structural units of the silicone polymer. For example, the silicon atom adjacent to the asterisk can be bonded to an additional oxygen atom, and the oxygen atom adjacent to the asterisk can be bonded to an additional silicon atom or else to a C₁-C₆ alkyl group.

A divalent C₁-C₂₀ alkylene group can alternatively also be termed a double-bond C₁-C₂₀ alkylene group, which means that each moiety ALK1 or ALK2 can have two bonds.

In the case of ALK1, the silicon atom is bonded to the moiety ALK1, and the second bond is between ALK1 and the secondary amino group.

In the case of ALK2, the secondary amino group bonds with the moiety ALK2, and the second bond is formed between ALK2 and the primary amino group.

Examples of a linear divalent C₁-C₂₀ alkylene group are, for example, the methylene group (—CH₂), the ethylene group (—CH₂—CH₂), the propylene group (—CH₂—CH₂—CH₂—) and the butylene group (—CH₂—CH₂—CH₂—CH₂—). The propylene group (—CH₂—CH₂—CH₂—) is particularly preferred. Starting at a chain length of 3 C atoms, divalent alkylene groups may also be branched. Examples of branched, divalent C₃-C₂₀ alkylene groups are (—CH₂—CH(CH₃)—) and (—CH₂—CH(CH₃)—CH₂—).

In an additional particularly preferred embodiment, the structural units of the formula (Si-amino) represent repeat units in the amino-functionalized silicone polymer (a3) so that the silicone polymer comprises several structural units of formula (Si-amino).

In the following, particularly well-suited amino-functionalized silicone polymers (a3) with at least one secondary amino group are listed.

Colorings with the greatest color intensities were obtainable when an agent was applied on the keratin material which contains at least one amino-functionalized silicone polymer (a3) which comprises structural units of formula (Si-I) and formula (Si-II):

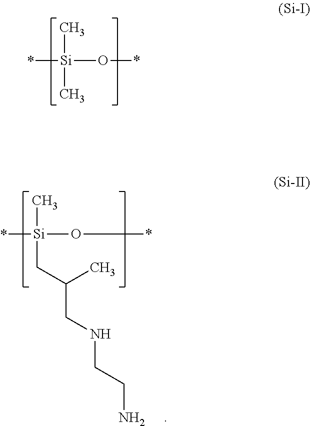

In an additional explicitly very particularly preferred embodiment, an agent according to the invention is characterized in that it contains at least one amino-functionalized silicone polymer (a3) which comprises structural units of formula (Si-I) and formula (Si-II):

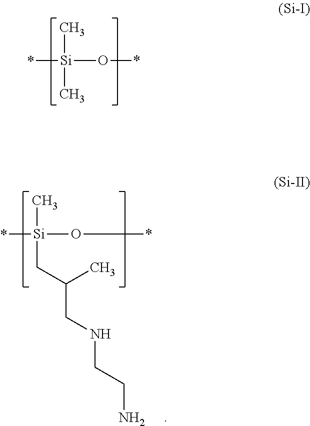

A corresponding amino-functionalized silicone polymer with the structural units (Si-I) and (Si-II) is, for example, the commercial product DC 2-8566 or Dowsil 2-8566 Amino Fluid which is sold commercially by Dow Chemical Company and which bears the designation “Siloxanes and Silicones, 3-[(2-Aminoethyl)amino]-2-methylpropyl Me, Di-Me-Siloxane” and the CAS number 106842-44-8. Another amino-functionalized silicone polymer with the structural units (Si-I) and (Si-II) is, for example, the commercial product DOWSIL™ AP-8568 Amino Fluid, which is likewise sold commercially by Dow Chemical Company.

In another preferred embodiment, an agent according to the invention is characterized in that it contains at least one amino-functional silicone polymer (a3) of formula of formula (Si-III):

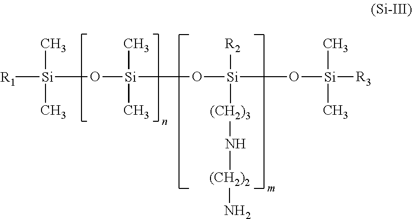

where
   m and n denote numbers that are selected such that the sum (n+m) is in a range of 1 to 1000,
   n is a number in a range from 0 to 999 and m is a number in a range from 1 to 1,000,
   R1, R2 and R3, which are identical or different, denote a hydroxyl group or a C₁₋₄ alkoxy group,
   at least one of the R1 to R3 groups denoting a hydroxyl group.

Additional agents preferred according to the invention are characterized by their content of at least one amino-functional silicone polymer (a3) of formula of formula (Si-IV),

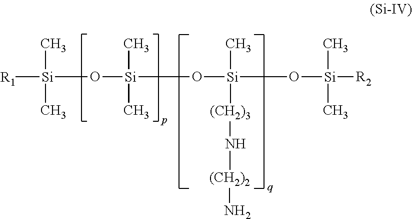

where
   p and q denote numbers that are selected such that the sum (p+q) is in a range from 1 to 1,000,
   p is a number in a range from 0 to 999 and q is a number in a range from 1 to 1,000, and
   R1 and R2, which are different, denote a hydroxy group or a C₁₋₄ alkoxy group, at least one of the groups R1 to R2 denoting a hydroxy group.

The silicones of formulas (Si-III) and (Si-IV) are different due to the grouping on the Si atom which carries the nitrogen-containing group. In formula (Si-III), R2 denotes a hydroxyl group or a C₁₋₄ alkoxy group while the functional group in formula (Si-IV) is a methyl group. The individual Si moieties, which are labeled with the indices m and n or p and q, need not be present as blocks; instead, the individual units can also be distributed randomly, i.e., in formulas (Si-III) and (Si-IV), each R1-Si(CH₃)₂ group is not necessarily bound to a —[O—Si(CH₃)₂] moiety.

Agents according to the invention which contain at least one amino-functional silicone polymer (a3) of the formula (Si-V) have also proven to be particularly effective with respect to the desired effects:

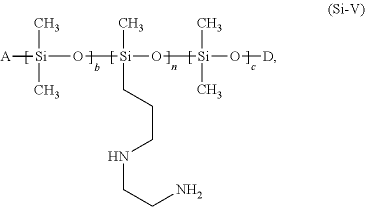

where
A represents an —OH, —O—Si(CH₃)₃, —O—Si(CH₃)₂OH, or —O—Si(CH₃)₂OCH₃ group,
D represents an —H, —Si(CH₃)₃, —Si(CH₃)₂OH, or —Si(CH₃)₂OCH₃ group,
b, n and c represent integers between 0 and 1,000,
with the proviso that
   n>0 and b+c>0
   at least one of the conditions A=—OH or D=—H is met.

In the above formula (Si-V), the individual siloxane units having the indices b, c and n are randomly distributed, i.e. they are not necessarily block copolymers.

The agent (a) can also comprise one or more different amino-functionalized silicone polymers which are described by formula (Si-VI):

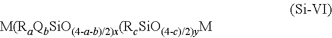

where, in the above formula, R is a hydrocarbon or a hydrocarbon group having 1 to approximately 6 carbon atoms, Q is a polar group of general formula -R¹HZ, where R¹ is a bivalent, linking group bonded to hydrogen and the group Z, composed of carbon and hydrogen atoms, carbon, hydrogen and oxygen atoms, or carbon, hydrogen and nitrogen atoms, and Z is an organic, aminofunctional group containing at least one aminofunctional group; “a” assumes values in a range of approximately 0 to approximately 2, “b” assumes values in a range of approximately 1 to approximately 3, “a”+“b” is less than or equal to 3, and “c” is a number in a range of approximately 1 to approximately 3, and x is a number in a range of 1 to approximately 2,000, preferably approximately 3 to approximately 50, and most preferably approximately 3 to approximately 25, and y is a number in a range of approximately 20 to approximately 10,000, preferably approximately 125 to approximately 10,000, and most preferably approximately 150 to approximately 1,000, and M is a suitable silicone end group as is known in the prior art, preferably trimethylsiloxy. Non-limiting examples of the groups represented by R include alkyl groups, such as methyl, ethyl, propyl, isopropyl, isopropyl, butyl, isobutyl, amyl, isoamyl, hexyl, isohexyl, and the like; alkenyl groups such as vinyl, halovinyl, alkylvinyl, allyl, haloallyl, and alkylallyl; cycloalkyl groups such as cyclobutyl, cyclopentyl, cyclohexyl, and the like; phenyl groups, benzyl groups, halohydrocarbon groups such as 3-chloropropyl, 4-bromobutyl, 3,3,3-trifluoropropyl, chlorocyclohexyl, bromophenyl, chlorophenyl, and the like, and sulfur-containing groups such as mercaptoethyl, mercaptopropyl, mercaptohexyl, mercaptophenyl, and the like; R is preferably an alkyl group, containing 1 to about 6 carbon atoms, and most preferably R is methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃CC(O)OCH₂CH₂—, —C₆H₄C₆H₄—, —C₆H₄CH₂C₆H₄—; and —(CH₂)₃C(O)SCH₂CH₂—.

Z is an organic, amino-functional group containing at least one functional amino group. A possible formula for Z is NH(CH₂)_{z}NH₂, where z is 1 or more. Another possible formula for Z is —NH(CH₂)_{z}(CH₂)_{zz}NH, in which both z and zz are independently 1 or more, wherein this structure comprises diamino ring structures, such as piperazinyl. Z is most preferably a —NHCH₂CH₂NH₂ group. Another possible formula for Z is N(CH₂)_{z}(CH₂)_{zz}NX₂ or —NX₂, where each X of X₂ is selected independently from the group consisting of hydrogen and alkyl groups having 1 to 12 carbon atoms, and zz is 0.

Q is most preferably a polar, amino-functional group of formula —CH₂CH₂CH₂NHCH₂CH₂NH₂. In the formulas, “a” assumes values in the range of about 0 to about 2, “b” assumes values in the range of about 2 to about 3, “a”+“b” is less than or equal to 3, and “c” is a number in the range of about 1 to about 3. The molar ratio of the RₐQ_{b}SiO_{(4-ab)/2} units to the R_{c}SiO_{(4-c)/2} units lies within a range of about 1:2 to 1:65, preferably about 1:5 to about 1:65 and most preferably about 1:15 to about 1:20. If one or more silicones of the above formula are used, then the various variable substituents in the above formula can be different in the various silicone components, present in the silicone mixture.

In another preferred embodiment, an agent according to the invention is characterized in that it contains at least one amino-functional silicone polymer of formula (Si-VII):

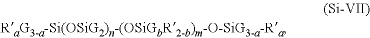

where:
   G is —H, a phenyl group, —OH, —O—CH₃, —CH₃, —O—CH₂CH₃, —CH₂CH₃, —O—CH₂CH₂CH₃, —CH₂CH₂CH₃, —O—CH(CH₃)₂, —CH(CH₃)₂, —O—CH₂CH₂CH₂CH₃, —CH₂CH₂CH₂CH₃, —O—CH₂CH(CH₃)₂, —CH₂CH(CH₃)₂, —O—CH(CH₃)CH₂CH₃, —CH(CH₃)CH₂CH₃, —O—C(CH₃)₃, or —C(CH₃)₃;
   a represents a number between 0 and 3, in particular 0;
   b represents a number between 0 and 1, in particular 1;
   m and n are numbers of which the sum (m+n) is between 1 and 2,000, preferably between 50 and 150, n preferably assuming values of 0 to 1,999 and in particular from 49 to 149 and m preferably assuming values of 1 to 2,000, in particular from 1 to 10;
   R′ is a monovalent group selected from
      -Q-N(R″)—CH₂—CH₂—N(R″)₂
      -Q-N(R″)₂
      -Q-N+(R″)₃A-
      -Q-N+H(R″)₂A-
      -Q-N+H₂(R″)A-
      -Q-N(R″)—CH₂—CH₂—N+R″H₂A-
where each Q represents a chemical bond, —CH₂—, —CH₂—CH₂—, —CH₂CH₂CH₂—, —C(CH₃)₂—, —CH₂CH₂CH₂CH₂—, —CH₂C(CH₃)₂—, or —CH(CH₃)CH₂CH₂—,
R″ represents identical or different functional groups from the group —H, -phenyl, -benzyl, —CH₂—CH(CH₃)Ph, from the C₁₋₂₀ alkyl groups, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, and A represents an anion preferably selected from chloride, bromide, iodide or methosulfate.

In another preferred embodiment, an agent according to the invention is characterized in that it contains at least one amino-functional silicone polymer (a3) of formula of (Si-VIIa):

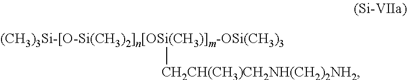

where m and n are numbers of which the sum (m+n) is between 1 and 2,000, preferably between 50 and 150, n preferably assuming values from 0 to 1,999 and in particular from 49 to 149 and m preferably assuming values from 1 to 2,000, in particular from 1 to 10.

These silicones are designated as trimethylsilylamodimethicones in accordance with the INCI declaration.

In another preferred embodiment, an agent according to the invention is characterized in that it contains at least one amino-functional silicone polymer of the formula (Si-VIIb):

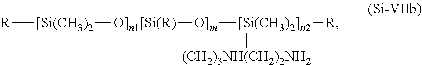

where R represents —OH, —O—CH₃, or a —CH₃ group and m, n1 and n2 are numbers of which the sum (m+n1+n2) amounts to between 1 and 2,000, preferably between 50 and 150, the sum (n1+n2) preferably assuming values from 0 to 1,999 and in particular from 49 to 149 and m preferably assuming values from 1 to 2,000, in particular from 1 to 10.

According to the INCI Declaration, these amino-functionalized silicone polymers are called amodimethicones.

Irrespective of which amino-functional silicones are used, agents according to the invention are preferred that contain an amino-functional silicone polymer (a3) of which the amine value is above 0.25 meq/g, preferably above 0.3 meq/g, and in particular above 0.4 meq/g. The amine value here represents the milliequivalents of amine per gram of the amino-functional silicone. Said value can be determined by titration and may also be given in the unit mg KOH/g.

Furthermore, agents which contained a specific 4-morpholinomethyl-substituted silicone polymer (a3) are also suitable. This amino-functionalized silicone polymer comprises structural units of formulas (Si-VIII) and of formula (Si-IX):

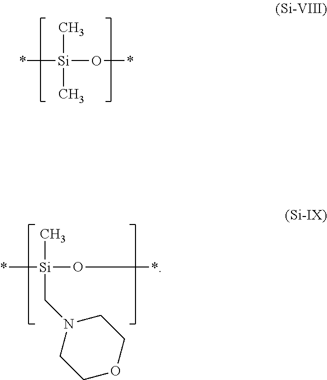

Corresponding 4-morpholinomethyl-substituted silicone polymers are described below.

A preferred amino-functionalized silicone polymer is known under the name of amodimethicone/morpholinomethyl silsesquioxane copolymer and is commercially available in the form of the raw material Belsil ADM 8301 E by Wacker.

For example, a silicone which has structural units of formulas (Si-VIII), (Si-IX) and (Si-X) can be used as 4-morpholinomethyl-substituted silicone:

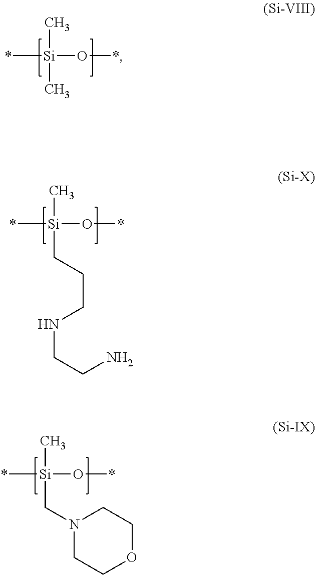

where
R1 represents —CH₃, —OH, —OCH₃, —O—CH₂CH₃, —O—CH₂CH₂CH₃, or —O—CH(CH₃)₂;
R2 represents —CH₃, —OH, or —OCH₃.

Particularly preferred agents according to the invention contain at least one 4-morpholinomethyl-substituted silicone of formula (Si-XI):

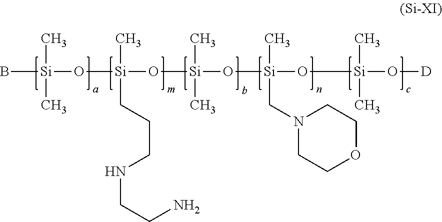

where
R1 represents —CH₃, —OH, —OCH₃, —O—CH₂CH₃, —O—CH₂CH₂CH₃, or —O—CH(CH₃)₂;
R2 represents —CH₃, —OH, or —OCH₃.
B represents an —OH, —O—Si(CH₃)₃, —O—Si(CH₃)₂OH, or —O—Si(CH₃)₂OCH₃ group,
D represents an —H, —Si(CH₃)₃, —Si(CH₃)₂OH, or —Si(CH₃)₂OCH₃ group,
a, b and c represent, independently of one another, integers between 0 and 1,000, with the proviso that a+b+c>0
m and n represent, independently of one another, integers between 1 and 1,000, with the proviso that
at least one of the conditions B=—OH or D=—H is met,
the units a, b, c, m and n are distributed randomly or in blocks in the molecule.

Structural formula (Si-XI) is intended to indicate that the siloxane groups n and m do not necessarily have to be directly bonded to an end group B or D. Instead, in preferred formulas (Si-VI), a>0 or b>0 and, in particularly preferred formulas (Si-VI), a>0 and c>0; i.e., the terminal group B or D is preferably bonded to a dimethylsiloxy group. In formula (Si-VI) as well, the siloxane units a, b, c, m and n are preferably distributed randomly.

The silicones represented by formula (Si-VI) and used according to the invention can be trimethylsilyl-terminated (D or B=—Si(CH₃)₃), but they may also be dimethylsilylhydroxy-terminated at both ends or dimethylsilylhydroxy- and dimethylsilylmethoxy-terminated at one end. Within the context of the present invention, silicones which are particularly preferably used are selected from silicones in which:
B=—O—Si(CH₃)₂OH and D=—Si(CH₃)₃
B=—O—Si(CH₃)₂OH and D=—Si(CH₃)₂OH
B=—O—Si(CH₃)₂OH and D=—Si(CH₃)₂OCH₃
B=—O—Si(CH₃)₃ and D=—Si(CH₃)₂OH
B=—O—Si(CH₃)₂OCH₃ and D=—Si(CH₃)₂OH.

These silicones lead to enormous improvements in the hair properties of hair treated with the agents according to the invention, and to greatly improved protection during oxidative treatment.

It has been found to be particularly advantageous if the agent according to the invention contains the amino-functionalized silicone polymer(s) (a3) in certain quantity ranges. Particularly good results were obtained when the agent contained, based on the total weight of the agent, a total amount from 0.1 to 8.0 wt. %, preferably 0.2 to 5.0 wt. %, more preferably from 0.3 to 3.0 wt. %, and very particularly preferably from 0.4 to 2.5 wt. %.

Within the scope of another particularly preferred embodiment, an agent according to the invention is characterized in that it contains, based on the total weight of the agent, one or more amino-functionalized silicone polymers (a3) in a total amount from 0.1 to 8.0 wt. %, preferably 0.2 to 5.0 wt. %, more preferably from 0.3 to 3.0 wt. %, and very particularly preferably from 0.4 to 2.5 wt. %.

Addition Products of C₁-C₆ Alkylene Oxide(s) to the Esters of C₁₂-C₃₀ Fatty Acids and Aromatic C₁-C₁₂ Alcohols (a4)

To further improve the fastness properties such as rub fastness and wash fastness, the agent according to the invention may additionally contain, as an optional component, at least one addition product of C₁-C₆ alkylene oxide(s) to the esters of C₁₂-C₃₀ fatty acids and C₁-C₁₂ aromatic alcohols.

The addition products of C₁-C₆ alkylene oxide(s) to the esters of C₁₂-C₃₀ fatty acids and aromatic C₁-C₁₂ alcohols (a4) are hereinafter also referred to as alkoxylated fatty acid esters (a4) for short.

C₁-C₆ alkylene oxides are the epoxides of C₁-C₆ alkanes. Examples of particularly well-suited C₁-C₆ alkylene oxides include ethylene oxide (1,2-epoxyethane), propylene oxide (1,2-epoxypropane) and butylene oxides (1,2-epoxybutane and 2,3-epoxybutane).

The ethoxylated fatty acid esters (a4) are based on C₁₂-C₃₀ fatty acids. These C₁₂-C₃₀ fatty acids according to the invention are linear or branched, saturated or mono- or polyunsaturated fatty acids which can also bear one or more hydroxyl groups. The C₁₂-C₂₄ fatty acids according to the invention are characterized in that they comprise 12 to 30 carbon atoms, preferably 12 to 24 carbon atoms. Furthermore, the C₁₂-C₂₄ fatty acids bear at least one carboxylic acid group.

To form the ethoxylated fatty acid esters (a4) according to the invention, for example one or more fatty acids can be used that are selected from the group consisting of dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z,12Z)-octadeca-9,12-dienoic acid], linolenic acid [(9Z,12Z,15Z)-octadeca-9,12,15-trienoic acid], elaeostearic acid [(9Z,11E,13E)-octadeca-9,11,3-trienoic acid], arachidonic acid [(5Z,8Z,11Z,14Z)-icosa-5,8,11,14-tetraenoic acid], nervonic acid [(15Z)-tetracos-15-enoic acid] and/or castor oleic acid ((9Z,12R)-12-hydroxy-9-octadecenoic acid).

The ethoxylated fatty acid esters (a4) are addition products of C₁-C₆ alkylene oxide(s) to the esters of the above-described C₁₂-C₂₄ fatty acids and aromatic C₁-C₁₂ alcohols. Characteristic of aromatic C₁-C₁₂ alcohols is that they comprise 1 to 12 carbon atoms and at least one aromatic ring system. The aromatic C₁-C₁₂ alcohols each have at least one hydroxy group which can either be located directly on the aromatic compound (such as, for example, in the case of phenol), or can be linked to the aromatic compound via an aliphatic unit (such as, for example, in the case of benzyl alcohol or 2-phenoxyethanol). The structures of the aromatic C₁-C₁₂ alcohols may also comprise additional heteroatoms such as oxygen or nitrogen.

The corresponding aromatic C₁-C₁₂ alcohols may be mono- or polyvalent alcohols, i.e., the alcohols may have one or more hydroxyl groups.

Monovalent aromatic C₁-C₁₂ alcohols are very particularly preferred. This compound class comprises exactly one hydroxy group. Suitable representatives may be, for example, phenol, benzyl alcohol, 2-phenylethyl alcohol and 2-phenoxyethanol.

Within the scope of another particularly preferred embodiment, an agent according to the invention is characterized in that it contains:
(a4) at least one addition product of C₁-C₆ alkylene oxide(s) to the esters of C₁₂-C₃₀ fatty acids and aromatic C₁-C₁₂ alcohols.

Within the scope of an additional embodiment, an agent for coloring keratin material, in particular human hair, is therefore preferred, containing:
(a1) at least alkylene glycol of formula (AG)

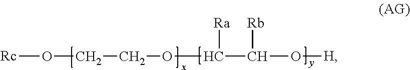

where
x represents an integer from 0 to 10,000,
y is an integer from 0 to 10,000,
Ra and Rb represent, independently of one another, a hydrogen atom, a C₁-C₆ alkyl group or a hydroxy-C₁-C₆ alkyl group,
Rc represents a hydrogen atom, a C₁-C₆ alkyl group, a phenyl group or a benzyl group, with the proviso that x+y is at least 1, and
(a2) at least one pigment, and
(a3) at least one amino-functionalized silicone polymer, and
(a4) at least one addition product of C₁-C₆ alkylene oxide(s) to the esters of C₁₂-C₃₀ fatty acids and monovalent aromatic C₁-C₁₂ alcohols.

Particularly good wash fastness and rub fastness was obtainable when, in the agent according to the invention, at least one addition product (a4) of C₁-C₆ alkylene oxide(s) to an ester was used which is obtained by esterification of a C₁₂-C₃₀ fatty acid having an aromatic C₁-C₁₂ alcohol from the group consisting of benzyl alcohol, phenol, 2-phenylethyl alcohol and 2-phenoxyethanol.

Within the scope of an additional particularly preferred embodiment, an agent according to the invention is characterized in that it contains:
(a4) at least one addition product of C₁-C₆ alkylene oxide(s) to an ester obtained by esterification of a C₁₂-C₂₄ fatty acid with a C₁-C₁₂ aromatic alcohol selected from the group consisting of benzyl alcohol, phenol, 2-phenylethyl alcohol and 2-phenoxyethanol, very particularly preferably benzyl alcohol.

Within the scope of a particularly preferred embodiment, an agent according to the invention is characterized in that it contains:
(a4) at least one addition product of C₁-C₆ alkylene oxide(s) to an ester which is obtained by esterification of a C₁₂-C₂₄ fatty acid with benzyl alcohol.

Within the scope of an additional particularly preferred embodiment, an agent according to the invention is characterized in that it contains:
(a4) comprises at least one addition product of ethylene oxide and/or propylene oxide to an ester which is obtained by esterification of a C₁₂-C₂₄ fatty acid having an aromatic C₁-C₁₂ alcohol from the group consisting of benzyl alcohol, phenol, 2-phenylethyl alcohol and 2-phenoxyethanol.

Within the scope of another particularly preferred embodiment, an agent according to the invention is characterized in that it contains:
(a4) comprises at least one addition product of ethylene oxide and/or propylene oxide to an ester which is obtained by esterification of a C₁₂-C₂₄ fatty acid with benzyl alcohol.

As already described above, ethylene oxide (1,2-epoxyethane), propylene oxide (1,2-epoxypropane) and butylene oxides (1,2-epoxybutane and 2,3-epoxybutane) can be used as particularly well-suited C₁-C₆ alkylene oxides. Ethylene oxide (1,2-epoxyethane) and propylene oxide (1,2-epoxypropane) are explicitly very particularly preferred. Propylene oxide (1,2-epoxypropane) is most preferred.

A corresponding addition product of ethylene oxide to the esters of C₁₂-C₃₀ fatty acids and aromatic C₁-C₁₂ alcohols (a4) arises when the C₁₂-C₃₀ fatty acid itself or the ester already formed therefrom is reacted with ethylene oxide (alternative name: 1,2-epoxyethane, CAS number 75-21-8).

Analogously, a corresponding addition product of propylene oxide to the esters of C₁₂-C₃₀ fatty acids and aromatic C₁-C₁₂ alcohols (a3) arises if the C₁₂-C₃₀ fatty acid itself or the ester already formed therefrom is reacted with propylene oxide (alternative name: 1,2-epoxypropane, CAS numbers 75-56-9 (racemate), 15448-47-2 ((R)-enantiomer, and 16088-62-3 (S)-enantiomer).

If the C₁₂-C₃₀ fatty acid itself reacts with ethylene oxide, initially an adduct can form starting from the carboxylic acid group of the C₁₂-C₃₀ fatty acid and the ethylene oxide, so that a *—C(O)—O—CH₂—CH₂—O—* group arises. This group is likewise an ester. If one mole of ethylene oxide is reacted per mole of fatty acid, a simple adduct with a *—CH2-CH2-O—* unit is formed in the agent. Depending on the molar excess used for the ethylene oxide, several adducts can also form, however, wherein there is a plurality of *—CH2-CH2-O—* units per mole C₁₂-C₃₀ fatty acid. To form the ester (a3), this adduct is then further reacted with at least one aromatic C₁-C₁₂ alcohol. The positions marked with an asterisk represent the bond to the remainder of the fatty acid and the bond to the remainder of the alcohol.

If the C₁₂-C₃₀ fatty acid is reacted analogously with propylene oxide, initially an adduct forms starting from the carboxylic acid group of the C₁₂-C₃₀ fatty acid and the propylene oxide, so that a *—C(O)—O—CH(CH)₃)—CH₂—O—* group or a *—C(O)—O—CH₂—CH(CH₃)—O—* group arises. A mixture of the two aforementioned moieties is usually obtained in the reaction mixture. Both moieties are likewise esters. If one mole of propylene oxide is reacted per mole of fatty acid, a simple adduct with a mixture of the units *—CH(CH₃)—CH₂—O—* and *—CH₂—CH(CH₃)—O—* is formed in the agent. Depending on the molar excess used for the propylene oxide, several adducts can also form, however, there is then a plurality of *CH(CH₃)—CH₂—O—* and/or *—CH₂—CH(CH₃)—O—* units per mole C₁₂-C₃₀ fatty acid. To form the ester (a3), this adduct is then further reacted with at least one aromatic C₁-C₁₂ alcohol. The positions marked with an asterisk represent the bond to the remainder of the fatty acid and the bond to the remainder of the alcohol.

Furthermore, it is conceivable in principle for the C₁₂-C₃₀ fatty acid to be reacted with a mixture of ethylene oxide and propylene oxide. In this case, mixtures of the adducts described above are formed. The reactions of higher alkylene oxides, for example butylene oxides, with the C₁₂-C₃₀ fatty acids are also thereby possible.

The addition products produced in this way lead, for example, to the alkoxylated fatty acid esters of the general formula (AFE-I)

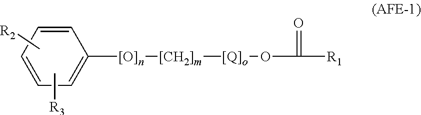

where
R1 represents a saturated or unsaturated C₁₁-C₂₉ alkyl group,
R2 and R3 represent, independently of one another, a hydrogen atom, a C₁-C₆ alkyl group,
a hydroxy group, or a C₁-C₆ alkoxy group.
n represents the number 0 or 1,
m represents an integer from 0 to 6,
o represents an integer from 1 to 60, and
Q represents a structural unit —O—CH₂—CH₂—, —O—CH(CH₃)—CH₂— or —O—CH₂—CH(CH₃)— with the proviso that if m is equal to 0, n is also equal to 0.

With agents containing at least ethoxylated fatty acid esters (a4) of formula (AFE-I), colorings were obtained which are particularly notable with respect to favorable wash fastness and rub fastness. For this reason, the use of one or more ethoxylated fatty acid esters (a4) of formula (AFE-I) in the agents as an additional component beyond components (a1), (a2) and (a3) is very particularly preferred.

Within the scope of another particularly preferred embodiment, an agent according to the invention is characterized in that it contains:
(a4) at least one alkoxylated fatty acid ester of the general formula (AFE-I)

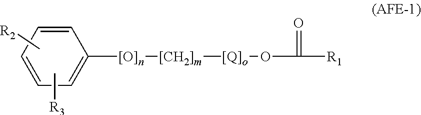

where
R1 represents a saturated or unsaturated C₁₁-C₂₉ alkyl group,
R2 and R3 represent, independently of one another, a hydrogen atom, a C₁-C₆ alkyl group,
a hydroxy group, or a C₁-C₆ alkoxy group.
n represents the number 0 or 1,
m represents an integer from 0 to 6,
o represents an integer from 1 to 60, and
Q represents a structural unit —O—CH₂—CH₂—, —O—CH(CH₃)—CH₂— or —O—CH₂—CH(CH₃)—
with the proviso that if m is equal to 0, n is also equal to 0.

The functional group R1 represents a saturated or unsaturated C₁₁-C₂₉ alkyl group. An unsaturated C₁₁-C₂₃ alkyl group can comprise one or more double bonds and is alternatively also as termed an unsaturated C₁₁-C₂₃ alkenyl group. The saturated or unsaturated C₁₁-C₂₉ alkyl group may be linear or branched.

Preferably, R1 represents a linear, saturated or unsaturated C₁₁-C₂₃ alkyl group.

The functional groups R2 and R3 are each a hydrogen atom, a C₁-C₆ alkyl group, a hydroxy group or a C₁-C₆ alkoxy group. Very particularly preferably, the functional groups R1 and R2 both represent a hydrogen atom.

The index number n is the number 0 or 1. Preferably, n is the number 0.

The index number m is an integer from 0 to 6. Preferably, m is the number 1.

The index number o is an integer from 1 to 60. Preferably, o is an integer from 1 to 30, more preferably 1 to 20, even more preferably 1 to 10, and very particularly preferably 1 to 5.

The Q functional group is a structural unit —O—CH₂—CH₂—, —O—CH(CH₃)—CH₂— or —O—CH₂—CH(CH₃)—. Particularly preferably, Q is a structural unit —O—CH(CH₃)—CH₂— or —O—CH₂—CH(CH₃)—.

If o is a number greater than 1, a plurality of structural units Q are present in the compounds of formula (AFE-I) (or also of formula (AFE-II), in which case each structural unit Q can be selected independently of the other structural units Q.

Accordingly, a preferred agent according to the invention is characterized in that it contains:
(a4) at least one alkoxylated fatty acid ester of general formula (AFE-I)

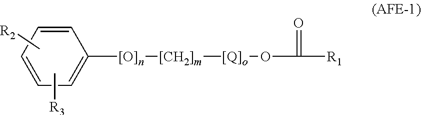

where
R1 represents a saturated or unsaturated C₁₁-C₂₉ alkyl group,
R2 and R3 represent, independently of one another, a hydrogen atom, a C₁-C₆ alkyl group,
a hydroxy group, or a C₁-C₆ alkoxy group.
n represents the number 0 or 1,
m represents an integer from 0 to 6,
o represents an integer from 1 to 60, and
Q represents a structural unit —O—CH₂—CH₂—, —O—CH(CH₃)—CH₂— or —O—CH₂—CH(CH₃)—
with the proviso that if m is equal to 0, n is also equal to 0, and wherein in the case that o is greater than 1, each structural unit Q can be selected independently of the other structural units Q.

In summary, particularly good results were obtained with the agents according to the invention which contained:
(a4) at least one alkoxylated fatty acid ester of the general formula (AFE-I),

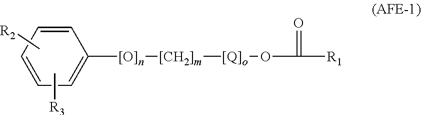

where
R1 represents a saturated or unsaturated C₁₁-C₂₉ alkyl group,
R2 and R3 both represent a hydrogen atom,
n represents the number 0,
m represents the number 1,
o represents an integer 1 to 30, preferably 1 to 20, more preferably 1 to 10, and very especially preferably 1 to 5, and
Q represents a structural unit —O—CH(CH₃)—CH₂— or —O—CH₂—CH(CH₃)—.

Within the scope of another very particularly preferred embodiment, an agent according to the invention is characterized in that it contains:
(a4) at least one alkoxylated fatty acid ester of the general formula (AFE-I)

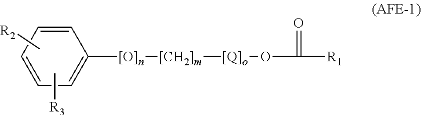

where
R1 represents a saturated or unsaturated C₁₁-C₂₉ alkyl group,
R2 and R3 both represent a hydrogen atom,
n represents the number 0,
m represents the number 1,
o represents an integer 1 to 30, preferably 1 to 20, more preferably 1 to 10, and very especially preferably 1 to 5, and
Q represents a structural unit —O—CH(CH₃)—CH₂— or —O—CH₂—CH(CH₃)—.

The very particularly preferred alkoxylated fatty acid esters of this embodiment also fall under the general formula (AFE-II)

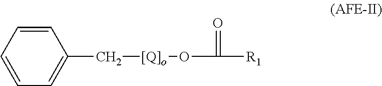

where
R1 represents a saturated or unsaturated C₁₁-C₂₉ alkyl chain,
o represents an integer 1 to 30, preferably 1 to 20, more preferably 1 to 10, and very especially preferably 1 to 5, and
Q represents a structural unit —O—CH(CH₃)—CH₂— or —O—CH₂—CH(CH₃)—.

Within the scope of another very particularly preferred embodiment, an agent according to the invention is characterized in that it contains:
(a4) at least one alkoxylated fatty acid ester of general formula (AFE-II)

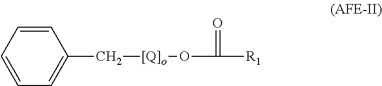

where
R¹ represents a saturated or unsaturated C₁₁-C₂₉ alkyl chain,
o represents an integer 1 to 30, preferably 1 to 20, more preferably 1 to 10, and very especially preferably 1 to 5, and
Q represents a structural unit —O—CH(CH₃)—CH₂— or —O—CH₂—CH(CH₃)—.

Here as well, in the case that o is greater than 1, each structural unit Q can be selected independently of the other structural units Q.

In this case, the structural units Q are oriented in the alkoxylated fatty acid esters of general formula (AFE-II) such that the oxygen atom in the group —O—CH(CH₃)—CH₂— or —O—CH₂—CH(CH₃)— is adjacent to the benzyl group, and the respective unit —CH2- or —CH(CH3)- is adjacent to the ester group —O—C(O)—R1.

An explicitly particularly well-suited compound of this type is PPG-3 benzyl ether myristate, which is alternatively also referred to as α-(1-oxotetradecyl)-ω-(phenylmethoxy) poly[oxy(methyl-1,2-ethanediyl)] and bears the CAS number 642443-86-5.

PPG-3 benzyl ether myristate can be purchased commercially, for example, under the trade name of Crodamol STS from Croda.

Within the scope of another very particularly preferred embodiment, an agent according to the invention is characterized in that it contains:
(a4) PPG-3 benzyl ether myristate.

The alkoxylated fatty acid esters (a4) are particularly preferably used within certain ranges of quality in the agent according to the invention.

Particularly good results were obtained when the agent contained, based on the total weight of the agent, one or more alkoxylated fatty acid esters (a4) in a total amount from 0.1 to 20.0 wt. %, preferably 0.5 to 15.0 wt. %, more preferably from 1.0 to 10.0 wt. %, still more preferably from 1.0 to 8.0 wt. %, and very particularly preferably from 1.0 to 5.0 wt. %.

Within the scope of another preferred embodiment, an agent according to the invention is
characterized in that it contains, based on the total weight of the agent, one or more alkoxylated fatty acid esters (a4) in a total amount from 0.1 to 20.0 wt. %, preferably 0.5 to 15.0 wt. %, more preferably from 1.0 to 10.0 wt. %, still more preferably from 1.0 to 8.0 wt. %, and very particularly preferably from 1.0 to 5.0 wt. %.

### Further Optional Components in the Agent

Depending on the desired form of production, the agent according to the invention can optionally also contain additional components or ingredients.

### Fatty Components in the Agent

If the agent is to be made available in the form of an emulsion or cream, the use of at least one fatty component has proven to be particularly advantageous. The fatty components are hydrophobic substances which can form emulsions in the presence of water while forming micelle systems. Without being committed to this theory, it is assumed that the C₁-C₆ alkoxysilanes, either in the form of their monomers or optionally in the form of their condensed oligomers, are embedded in this hydrophobic environment or in the micelle systems so that the polarity of their surroundings changes. Due to the hydrophobic character of the fatty components, the environment of the C₁-C₆ alkoxysilanes is also hydrophobicized. It is assumed that the polymerization reaction of the C₁-C₆ alkoxysilanes yielding the film or coating occurs in a milieu with a reduced polarity at reduced speed.

“Fatty components,” within the context of the invention are understood to be organic compounds with a solubility in water of less than 1 wt. %, and preferably less than 0.1 wt. % at room temperature (22° C.) and atmospheric pressure (760 mmHg). The definition of fatty components also explicitly includes only uncharged (i.e. non-ionic) compounds. Fat components have at least a saturated or unsaturated alkyl group with at least 12 carbon atoms. The molecular weight of the fatty component is at most 5,000 g/mol, preferably at most 2,500 g/mol, and particularly preferably at most 1,000 g/mol. The fatty components are either polyoxyalkylated or polyglycerylated compounds.

Very particularly preferably, the fatty components additionally used in the agent can be selected from the group consisting of C₁₂-C₃₀ fatty alcohols, C₁₂-C₃₀ fatty acid triglycerides, C₁₂-C₃₀ fatty acid monoglycerides, C₁₂-C₃₀ fatty acid diglycerides and/or hydrocarbons.

Within the scope of another preferred embodiment, an agent according to the invention is characterized in that it contains one or more fatty components from the group consisting of C₁₂-C₃₀ fatty alcohols, C₁₂-C₃₀ fatty acid triglycerides, C₁₂-C₃₀ fatty acid monoglycerides, C₁₂-C₃₀ fatty acid diglycerides and/or hydrocarbons.

The components from the group consisting of C₁₂-C₃₀ fatty alcohols, C₁₂-C₃₀ fatty acid triglycerides, C₁₂-C₃₀ fatty acid monoglycerides, C₁₂-C₃₀ fatty acid diglycerides and/or hydrocarbons are understood in this context to be very particularly preferred fatty components. In the sense of the present invention, only non-ionic substances are explicitly considered as fatty components. Charged compounds, such as fatty acids and salts thereof, are not understood to be fatty components.

C₁₂-C₃₀ fatty alcohols can be saturated, mono- or polyunsaturated, linear or branched fatty alcohols having 12 to 30 C atoms.

Examples of preferred linear saturated C₁₂-C₃₀ fatty alcohols are dodecan-1-ol (dodecyl alcohol, lauryl alcohol), tetradecan-1-ol (tetradecyl alcohol, myristyl alcohol), hexadecan-1-ol (hexadecyl alcohol, cetyl alcohol, palmityl alcohol), octadecan-1-ol (octadecyl alcohol, stearyl alcohol), arachyl alcohol (eicosan-1-ol), heneicosyl alcohol (heneicosan-1-ol) and/or behenyl alcohol (docosan-1-ol).

Preferred linear unsaturated fatty alcohols are (9Z)-octadec-9-en-1-ol (oleyl alcohol), (9E)-octadec-9-en-1-ol (elaidyl alcohol), (9Z,12Z)-octadeca-9,12-dien-1-ol (linoleyl alcohol), (9Z,12Z,Z)-octadeca-9,12, 15-trien-1-ol (linolenoyl alcohol), gadoleyl alcohol ((9Z)-eicos-9-en-1-ol), arachidone alcohol ((5Z,8Z,11Z,14Z)-eicosa-5,8,11,14-tetraen-1-ol), erucyl alcohol ((13Z)-docos-13-en-1-ol), and/or brassidyl alcohol ((13E)-docosen-1-ol).

The preferred representatives of branched fatty alcohols are 2-octyl-dodecanol, 2-hexyl dodecanol and/or 2-butyl-dodecanol.

Within the scope of another preferred embodiment, an agent according to the invention is characterized in that it contains one or more C₁₂-C₃₀ fatty alcohols (a4) from the group consisting of
dodecan-1-ol (dodecyl alcohol, lauryl alcohol),
tetradecan-1-ol (tetradecyl alcohol, myristyl alcohol),
hexadecan-1-ol (hexadecyl alcohol, cetyl alcohol, palmityl alcohol),
octadecan-1-ol (octadecyl alcohol, stearyl alcohol),
arachyl alcohol (eicosan-1-ol),
heneicosyl alcohol (heneicosan-1-ol),
behenyl alcohol (docosan-1-ol).
(9Z)-octadec-9-en-1-ol (oleyl alcohol),
(9E)-octadec-9-en-1-ol (elaidyl alcohol),
(9Z,12Z)-octadeca-9,12-dien-1-ol (linoleyl alcohol),
(9Z,12Z,15Z)-octadeca-9,12,15-trien-1-ol (linolenyl alcohol),
gadoleyl alcohol (9Z)-eicos-9-en-1-ol),
arachidone alcohol (5Z,8Z,11Z,14Z)-eicosa-5,8,11,14-tetraen-1-ol),
erucyl alcohol (13Z)-docos-13-en-1-ol),
brassidyl alcohol (13E)-docosen-1-ol),
2-octyldodecanol,
2-hexyldodecanol and/or
2-butyldodecanol.

It has proven to be very particularly preferred to use one or more C₁₂-C₃₀ fatty alcohols within very specific quantity ranges.

Furthermore, it is particularly preferred if the agent, based on the total weight of the agent, contains one or more C₁₂-C₃₀ fatty alcohols in a total amount from 2.0 to 50.0 wt. %, preferably from 3.0 to 30.0 wt. %, more preferably from 4.0 to 20.0 wt. %, still more preferably from 5.0 to 15.0 wt. %, and very particularly preferably from 5.0 to 10.0 wt. %.

Furthermore, the agent may also contain at least one C₁₂-C₃₀ fatty acid triglyceride, the C₁₂-C₃₀ fatty acid monoglyceride and/or C₁₂-C₃₀ fatty acid diglyceride as a wholly suitable fatty component. For a C₁₂-C₃₀ fatty acid triglyceride, in the context of the present invention, the triesters of trivalent alcohol glycerol are understood with three equivalents of fatty acid. Both structurally similar and different fatty acids may be involved in the ester formation within a triglyceride molecule.

For fatty acids, according to the invention, saturated or unsaturated, unbranched or branched, unsubstituted or substituted C₁₂-C₃₀-carboxylic acids are to be understood. Unsaturated fatty acids may be mono-unsaturated or poly-unsaturated. With an unsaturated fatty acid, the C—C double bond(s) thereof may have the cis or trans configuration.

Notable for particular suitability are fatty acid triglycerides in which at least one of the ester groups originating from glycerol is formed with a fatty acid, selected from dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z,12Z)-octadeca-9,12-dienoic acid], linolenic acid [(9Z,12Z,15Z)-octadeca-9,12,15-trienoic acid], eleostearic acid [(9Z,11E,13E)-octadeca-9,11,3-trienoic acid], arachidonic acid [(5Z,8Z,11Z,14Z)-eicosa-5,8,11,14-tetraenoic acid], and/or nervonic acid [(15Z)-tetracos-15-enoic acid].

The fatty acid triglycerides may also be of natural origin. The fatty acid triglycerides or mixtures thereof derived from soybean oil, groundnut oil, olive oil, sunflower oil, macadamia nut oil, moringa oil, apricot kernel oil, marula oil and/or optionally hydrogenated castor oil are particularly suitable for use in the products according to the present invention.

A C₁₂-C₃₀ fatty acid monoglyceride is understood to be the monoester of trivalent alcohol glycerol with a fatty acid equivalent. In this case, either the middle hydroxyl group of glycerol or the terminal hydroxyl group of glycerol may be esterified with a fatty acid.

Preferred C₁₂-C₃₀ fatty acid esters are the esters, which are formed by esterification of the alcohols ethanol and/or isopropanol with one of the fatty acids from the group of dodecanoic acid (lauric acid), tetradecanoic acid (myristic acid), hexadecanoic acid (palmitic acid), tetracosanoic acid (lignoceric acid), octadecanoic (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z,12Z)-octadeca-9,12-dienoic acid], linolenic acid [(9Z,12Z,15Z)-octadeca-9,12,15-trienoic acid], eleostearic acid [(9Z,11E, 13E)-octadeca-9,11,3-trienoic acid], arachidonic acid [(5Z,8Z,11Z,14Z)-icosa-5,8,11,14-tetra acid] and/or nervonic acid [(15Z)-tetracos-15-enoic acid].

A C₁₂-C₃₀ fatty acid diglyceride is understood to mean the diester of the trivalent alcohol glycerol with two equivalents of fatty acid. In this case, either the middle and a terminal hydroxy group of glycerol can be esterified with two fatty acid equivalents, or however both terminal hydroxy groups of glycerol are each esterified with one fatty acid. Glycerol can be esterified hereby both with two structurally similar and with two different fatty acids.

The fatty acid diglycerides are characterized by a particular suitability in which at least one of the ester groups, starting from glycerol, is formed with a fatty acid, which is selected from dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z, 12Z)-octadeca-9,12-dienoic acid], linolenic acid [(9Z,12Z,15Z)-octadeca-9,12,15-trienoic acid], eleostearic acid [(9Z,11E, 13E)-octadeca-9,11,3-trienoic acid], arachidonic acid [(5Z,8Z,11Z,14Z)-icosa-5,8,11,14-tetraenoic acid] and/or nervonic acid [(15Z)-tetracos-15-enoic acid].

Particularly good results were obtained when the composition contained at least one C₁₂-C₃₀ fatty acid monoglyceride selected from the monoesters of glycerol with a fatty acid equivalent from the group consisting of dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z,12Z)-octadeca-9,12-dienoic acid], linolenic acid [(9Z,12Z,15Z)-octadeca-9,12,15-trienoic acid], eleostearic acid [(9Z,11E, 13E)-octadeca-9,11,3-trienoic acid], arachidonic acid [(5Z,8Z,11Z,14Z)-icosa-5,8,11,14-tetraenoic acid] and/or nervonic acid [(15Z)-tetracos-15-enoic acid].

Within the scope of another embodiment, an agent according to the invention is characterized in that it contains at least one C₁₂-C₃₀ fatty acid monoglyceride which is selected from the monoesters of glycerol with a fatty acid equivalent from the group consisting of dodecanoic acid, tetradecanoic acid, hexadecanoic acid, tetracosanoic acid, octadecanoic acid, eicosanoic acid and/or docosanoic acid.

It has been shown to be preferable to use one or more C₁₂-C₃₀ fatty acid monoglycerides, C₁₂-C₃₀ fatty acid diglycerides and/or C₁₂-C₃₀ fatty acid triglycerides (a4) in very specific ranges of amounts in the composition.

With regard to the solution of the task according to the invention, it has proven advantageous if the agent, based on the total weight of the agent, contains one or more C₁₂-C₃₀ fatty acid monoglycerides, C₁₂-C₃₀ fatty acid diglycerides and/or C₁₂-C₃₀ fatty acid triglycerides (a4) in a total amount from 0.1 to 20.0 wt. %, preferably 0.3 to 15.0 wt. %, more preferably 0.5 to 10.0 wt. %, and very particularly preferably from 0.8 to 5.0 wt. %.

Within the scope of a very particularly preferred embodiment, a method according to the invention is characterized in that the agent, based on the total weight of the agent, is one or more C₁₂-C₃₀ fatty acid monoglycerides, C₁₂-C₃₀ fatty acid diglycerides and/or C₁₂-C₃₀ fatty acid triglycerides in a total amount from 0.1 to 20.0 wt. %, preferably 0.3 to 15.0 wt. %, more preferably 0.5 to 10.0 wt. %, and very particularly preferably from 0.8 to 5.0 wt. %.

The C₁₂-C₃₀ fatty acid monoglycerides, C₁₂-C₃₀ fatty acid diglycerides and/or C₁₂-C₃₀ fatty acid triglycerides can be used as the sole fatty components (a4) in the agents. However, it may also be possible according to the invention to incorporate at least one C₁₂-C₃₀ fatty acid monoglyceride, C₁₂-C₃₀ fatty acid diglyceride and/or C₁₂-C₃₀ fatty acid triglyceride in combination with at least one C₁₂-C₃₀ fatty alcohol into the composition.

Furthermore, as a very particularly preferred fat component, the composition may also contain at least one hydrocarbon.

Hydrocarbons are compounds have 8 to 80 C atoms composed exclusively of carbon and hydrogen atoms. Especially preferred in this context are aliphatic hydrocarbons, such as mineral oils, liquid paraffin oils (e.g., paraffinum liquidum or paraffinum perliquidum), isoparaffin oils, semisolid paraffin oils, paraffin waxes, solid paraffin (paraffinum solidum), Vaseline, and polydecene.

In this respect, liquid paraffin oils (Paraffinum Liquidum and Paraffinum Perliquidum) have proven to be particularly suitable. The hydrocarbon is very particularly preferably Paraffinum Liquidum, also referred to as white oil.

Paraffinum Liquidum is a mixture of purified, saturated, aliphatic hydrocarbons, consisting mainly of hydrocarbon chains having a C-chain distribution of from 25 to 35 C atoms.

Very particularly good results were obtained when the agent contained at least one hydrocarbon selected from the group consisting of mineral oils, the liquid paraffin oils, isoparaffin oils, semisolid paraffin oils, paraffin waxes, hard paraffin (Paraffinum solidum), Vaseline and polydecenes.

Within the scope of very particularly preferred embodiment, an agent according to the invention is characterized in that it contains at least one fatty component from the group of hydrocarbons.

With regard to the solution of the problem according to the invention, it proved to be very particularly preferable if the composition contained, based on the total weight of the composition, one or more hydrocarbons in a total amount from 0.5 to 20.0 wt. %, preferably 0.7 to 10.0 wt. %, more preferably from 0.9 to 5.0 wt. %, and very particularly preferably from 1.0 to 4.0 wt. %.

### Additional Ingredients in the Agent

The agents may also contain other active ingredients, auxiliaries and additives such as structurants like glucose, maleic acid and lactic acid, hair-conditioning compounds like phospholipids, for example lecithin and kephalins; perfume oils, dimethyl isosorbide and cyclodextrins; polymers like anionic, nonionic and cationic polymers; surfactants such as anionic, nonionic, cationic, zwitterionic and amphoteric surfactants, fatty components, fiber structure-improving active ingredients, in particular mono-, di- and oligosaccharides such as glucose, galactose, fructose, fructose and lactose; dyes for coloring the agent; anti-dandruff active ingredients such as piroctone olamine, zinc omadine and climbazole; amino acids and oligopeptides; protein hydrolysates based on animals and/or vegetables, as well as in the form of their fatty acid condensation products or optionally anionically or cationically modified derivatives; vegetable oils; light stabilizers and UV blockers; active ingredients such as panthenol, pantothenic acid, pantolactone, allantoin, pyrrolidinonecarboxylic acids and their salts, and bisabolol; polyphenols, in particular hydroxycinnamic acids, 6,7-dihydroxycoumarins, hydroxybenzoic acids, catechins, tannins, leucoanthocyanidins, anthocyanidins, flavanones, flavones and flavonols; ceramides or pseudoceramides; vitamins, provitamins and vitamin precursors; plant extracts; fats and waxes such as fatty alcohols, beeswax, montan wax and paraffins; swelling and penetrating agents such as glycerol, propylene glycol monoethyl ether, carbonates, hydrogen carbonates, guanidines, ureas and primary, secondary and tertiary phosphates; opacifiers such as latex, styrene/PVP and styrene/acrylamide copolymers; pearlescent agents such as ethylene glycol mono- and distearate and PEG-3 distearate; and blowing agents such as propane-butane mixtures, N₂O, dimethyl ether, CO₂ and air.

The selection of these additional substances is made by the skilled artisan according to the desired properties of the agents. In regard to other facultative components and the employed amounts of said components, reference is made expressly to relevant handbooks known to the skilled artisan. The additional active ingredients and auxiliaries are used in the preparations according the invention preferably always in amounts from 0.0001 to 25 wt. %, in particular from 0.0005 to 15 wt. %, relative to the total weight of the particular agent.

### Methods for Dyeing Keratin Materials

The agents described above can be used outstandingly in methods for dyeing keratin material, in particular human hair.

A second object of the present invention is therefore a method for dyeing keratin material, in particular human hair, in which an agent as disclosed in detail in the description of the first object of the invention is applied to the keratin fibers and, if necessary, rinsed out again after an exposure time of 30 seconds to 45 minutes.

In other words, a second object of the invention is a method for dyeing keratin material, in particular human hair, comprising the following steps:
(1) applying a coloring agent on the keratin material, wherein the coloring agent is an agent as has been disclosed in detail in the description of the first object of the invention,
(2) exposing the coloring agent to the keratin material, and
(3) rinsing out the coloring agent with water.

In step (1) of the method according to the invention, the agent of the first object of the invention is applied to the keratin material, which is very particularly preferably human hair.

In step (2) of the method according to the invention, the agent is then allowed to act on the keratin material after its application. In this context, various exposure times of, for example, 30 seconds to 60 minutes are conceivable.

However, a great advantage of the dyeing system according to the invention is that an intensive color result can be achieved even in very short periods after short exposure times. For this reason, it is advantageous for the application mixture to remain on the keratin material after application only for comparatively short periods of 30 seconds to 15 minutes, preferably 30 seconds to 10 minutes, and particularly preferably 1 to 5 minutes.

In another preferred embodiment, a method according to the invention is characterized by
(2) exposing the keratin material to the coloring agent for a period of 30 seconds to 15 minutes, preferably 30 seconds to 10 minutes, and more preferably 1 to 5 minutes.

After the keratin material has been exposed to the application mixture, the keratin material is rinsed with water in step (3) of the process.

In one embodiment, the application mixture can be washed out with water only, i.e., without the aid of an after-treatment agent or a shampoo. The use of an after-treatment agent or conditioner in step (6) is also conceivable in principle.

To achieve the object according to the invention and to increase the application comfort, it has, however, been found to be very particularly preferred to rinse out the agent in step (3) exclusively with water without the aid of an additional after-treatment agent, shampoos or conditioners.

In another preferred embodiment, a method according to the invention is characterized by
(3) rinsing out the coloring agent exclusively with water.
   Method for Dyeing Keratin Material in which the Ready-to-Use Agent is First Produced.

As previously described, the agent of the first object of the invention is an agent ready for use which is either provided directly to the user in its ready-to-use form, or which is prepared by mixing various agents just prior to use.

In order to ensure a particularly fine distribution of the pigments, it has been found to be very particularly preferred to produce the ready-to-use agent shortly before application by mixing two or three different agents.

Within the scope of a particularly preferred embodiment, the ready-to-use agent is accordingly prepared by mixing at least two different agents, wherein the first of these two agents comprises the mixture of at least alkylene glycol of formula (AG) (a1) and pigment(s) (a2). For example, the mixture of alkylene glycol of formula (AG) (a1) and pigment(s) (a2) can represent a predispersion. The second agent contains at least one amino-functionalized silicone polymer (a3). To produce the ready-to-use agent, the two aforementioned agents are then shaken or stirred with each other.

Another object of the present application is therefore a method for coloring keratin material, in particular human hair, comprising the following steps:
(1) providing an anhydrous agent (I), wherein the agent (I) contains:
   (a1) at least one alkylene glycol of formula (AG) and
   (a2) at least pigment,
(2) providing an anhydrous agent (II), wherein the agent (II) contains:
   (a3) at least one amino-functionalized silicone polymer.
(3) producing an application mixture by mixing the agents (I) and (II),
(4) applying the application mixture produced in step (3) to the keratin material,
(5) exposing the keratin material to the application mixture applied in step (4), and
(6) rinsing out the application mixture with water,
wherein the ingredients (a1), (a2) and (a3) have already been disclosed in detail in the description of the first object of the invention.

The agent (I) is preferably a predispersion of the pigments (a2) in the alkylene glycol(s) of formula (AG) (a1) which can be in the form of a concentrate, for example.

If the agents (I) and (II) are mixed, the predispersion of the pigments (a2) in the alkylene glycol of formula (AG) (a1) ensures a particularly fine distribution of the pigments which is maintained even after the two agents (I) and (II) are mixed in the ready-to-use agent.

Finally, in the context of an additional embodiment, it can also prove advantageous to separate the components (a1) and (a2) from one another, so that the three components (a1), (a2) and (a3) which are previously separated from one another are mixed with each other during the preparation of the ready-to-use agent. This form of production can be advantageous, for example, when the pigment(s) (a2) are to be used in smaller amounts and/or in the form of a powder. The preparation of the pigments in powder form simplifies the quantitative transfer of the pigments into a mixing vessel or another container.

Another object of the present application is therefore a method for coloring keratin material, in particular human hair, comprising the following steps:
(1) providing an anhydrous agent (I), wherein the agent (I) contains:
   (a1) at least one alkylene glycol of formula (AG),
(2) providing an anhydrous agent (II), wherein the agent (II) contains:
   (a2) at least pigment,
(3) providing an anhydrous agent (III), wherein the agent (III) contains:
   (a3) at least one amino-functionalized silicone polymer,
(4) preparing an application mixture by mixing agents (I) and (II) and (III),
(5) applying the application mixture produced in step (4) to the keratin material,
(6) exposing the keratin material to the application mixture applied in step (5), and
(7) rinsing out the application mixture with water,
wherein the ingredients (a1), (a2) and (a3) have already been disclosed in detail in the description of the first object of the invention.

In this case, the agent (I) contains at least one alkylene glycol of formula (AG) (a1) and can be present, for example, in the form of a concentrate.

The agent (II) contains at least one pigment (a2). In one possible type of production, the pigment is provided, for example, in the form of a powder.

The agent (III) contains at least one amino-functionalized silicone polymer (a3) and is preferably present in the form of a concentrate.

### Multicomponent Packaging Unit

To increase user comfort, the above-described agents in the form of a multicomponent packaging unit can be provided to the user.

Another object is therefore a multicomponent packaging unit (kit of parts) for dyeing keratin material, in particular human hair, comprising separately prepared:
a first container with an anhydrous agent (I), wherein the agent (I) contains:
   (a1) at least one alkylene glycol of formula (AG) and
   (a2) at least pigment,
a second container with an anhydrous agent (II), wherein the agent (II) contains:
   (a3) at least one amino-functionalized silicone polymer,
wherein the ingredients (a1), (a2) and (a3) have already been disclosed in detail in the description of the first object of the invention.

Another object of the present invention is therefore a multicomponent packaging unit (kit of parts) for dyeing keratin material, in particular human hair, comprising separately prepared:
a first container with an anhydrous agent (I), wherein the agent (I) contains:
   (a1) at least one alkylene glycol of formula (AG),
a second container with an anhydrous agent (II), wherein the agent (II) contains:
   (a2) at least pigment,
a third container with an anhydrous agent (III); wherein the agent (III) contains:
   (a3) at least one amino-functionalized silicone polymer,
wherein the ingredients (a1), (a2) and (a3) have already been disclosed in detail in the description of the first object of the invention.

Concerning the additional preferred embodiments of the methods according to the invention and the multicomponent packaging unit according to the invention, what has been said about the agent according to the invention applies mutatis mutantis.

### EXAMPLES

### 1. Formulations

The following ready-to-use coloring agents were prepared (all data are in percent by weight unless stated otherwise):

| Ready-to-use coloring agent | E1 | E2 |
|---|---|---|
| PPG-3 benzyl ether myristate (Crodamol STS) (a4) | 3.40 | 3.40 |
| PPG-14 butyl ether (UCON FLUID AP, Dow Corning) | — | 1.00 |
| (a13) | | |
| Unipure Red LC 3071 (CI 15850, Sensient) (a2) | 0.38 | 0.38 |
| Aminosilicone (Dow Corning 2-8566 (Siloxanes and | 0.74 | 0.74 |
| Silicones, 3-[(2-Aminoethyl)amino]-2-methylpropyl | | |
| Me, Di-Me-Siloxane) (a3) | | |
| PEG 6000 (a12) (polyethylene glycol, molecular weight | 9.93 | 9.93 |
| 6000 to 7500 g/mol) | | |
| PEG 400 (a11) (polyethylene glycol, molecular weight | up to | up to |
| 400 g/mol) | 100 | 100 |

### 2. Application

The ready-to-use agents E1 and E2 produced beforehand were each applied to hair strands (Kerling, type “Euronatur hair white” (ENH)) (liquor ratio: 1 g agent per g of hair strand) and allowed to act for three minutes. Subsequently, the hair strands were washed thoroughly (1 minute) with water and dried.

### 3. Measurement of Wash Fastness

After drying, the colored strands were measured using a colorimeter from Datacolor, type Spectraflash 450.

Each strand was then washed three times manually. To this end, each strand was moistened with water, then a commercial shampoo (Schauma 7-Krauter) was applied to the strand (0.25 g shampoo per 1 g of hair) and massaged with the fingers 30 seconds. The strand was then rinsed for 1 minute under running lukewarm water, and the strand of hair was dried. The process described above corresponds to hair washing. For each additional hair wash, the process was repeated. The strands were colorimetrically measured again after 3 hair washes and after 6 hair washes.

The dE value used for the assessment of wash fastness is derived from the L*a*b* colorimetric values measured from the respective strand as follows:

$$dE = {[ {( {L}_{i} - {L}_{0} )}^{2} + {( {a}_{i} - {a}_{0} )}^{2} + ( {b}_{i} - {b}_{0} ) ]}^{1 / 2}$$

L₀, a₀ and b₀=measured values directly after dyeing
Lᵢ, aᵢ and bᵢ=measured values after 3 hair washes (and after 6 hair washes)

The smaller the dE value, the lesser the color difference compared to the dyed, unwashed hair, and the better the wash fastness.

FA describes the percent color content after the corresponding number of hair washes and is calculated according to the following formula:

$$FA [ \% ] = 100 * ( \frac{Δ E ( u - g )}{Δ E ( u - c )} )$$

FA=color retention in [%]
u=untreated starting hair
g=washed colored hair
c=colored hair

The higher the color content, the better the wash fastness.

| | E1 | E2 |
|---|---|---|
| dE (uncolored versus colored) | 60.40 | 61.10 |
| dE (colored, after 3 hair washes) | 1.20 | 3.40 |
| FA after 3 hair washes [%] | 100.00 | 105.00 |
| dE (colored, after 6 hair washes) | 1.20 | 6.30 |
| FA after 6 hair washes [%] | 102.00 | 98.00 |

With the agents E1 and E2, the hair strands were able to be intensively colored and had a very good wash fastness and a high color content.

1. An anhydrous agent for dyeing keratin material, wherein the agent comprises:
at least one alkylene glycol of formula (AG) 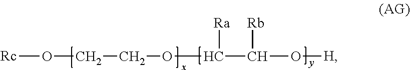
where
x represents an integer ranging from 0 to 10,000;
y represents an integer ranging from 0 to 10,000;
Ra and Rb represent, independently of one another, a hydrogen atom, a C₁-C₆ alkyl group or a hydroxy-C₁-C₆ alkyl group,
Rc represents a hydrogen atom, a C₁-C₆ alkyl group, a phenyl group, or a benzyl group;
wherein x+y is at least 1;
at least one pigment; and
at least one amino-functionalized silicone polymer.

2. The agent according to claim 1,
wherein:
x represents an integer ranging from 1 to 10,000;
y represents the number 0; and
Rc represents a hydrogen atom.

3. The agent according to claim 2,
wherein x is an integer ranging from 1 to 100.

4. The agent according to claim 2,
wherein x is an integer ranging from 101 to 1,000.

5. The agent according to claim 1,
wherein x is 0, and wherein y represents an integer ranging from 1 to 1,000;
Ra and Rb represent, independently of one another, a hydrogen atom or a C₁-C₆ alkyl group; and
Rc′ represents a C₁-C₆ alkyl group.

6. The agent according to claim 1, wherein, based on the total weight of the agent, one or more alkylene glycols of formula (AG) are present in a total amount ranging from 10.0 to 99.0 wt.

7. The agent according to claim 1, wherein, based on the total weight of the agent, the one or more alkylene glycols of formula (AG) comprise:
a first alkylene glycol where x represents an integer ranging from 1 to 10,000, y is 0, and Rc represents a hydrogen atom; wherein the first alkylene glycol is present in a total amount ranging from 20.0 to 99.0 wt. %;
a second alkylene glycol where x represents an integer ranging from 101 to 1,000, y represents the number 0, and Rc represents a hydrogen atom, wherein the second alkylene glycol is present in a total amount ranging from 1.0 to 35.0 wt. %;
a third alkylene glycol where x is 0, y represents an integer ranging from 1 to 1,000;
wherein the third alkylene glycol is present in a total amount ranging from 0.1 to 20.0 wt. %; or
combinations thereof.

8. The agent according to claim 1, wherein the at least one pigment is selected from the group comprising colored metal oxides, metal hydroxides, metal oxide hydrates, silicates, metal sulfides, complex metal cyanides, metal sulphates, bronze pigments, and/or from mica-based colored pigments which are coated with at least one metal oxide and/or a metal oxychloride, or combinations thereof.

9. The agent according to claim 1, wherein the at least one organic pigment is selected from the group comprising:
carmine;
quinacridone;
phthalocyanine;
sorghum;
blue pigments with the Color Index numbers CI 42090, CI 69800, CI 69825, CI 73000, CI 74100, CI 74160, or combinations thereof;
yellow pigments with the Color Index numbers CI 11680, CI 11710, CI 15985, CI 19140, CI 20040, CI 21100, CI 21108, CI 47000, CI 47005, or combinations thereof;
green pigments with the Color Index numbers CI 61565, CI 61570, CI 74260, or combinations thereof;
orange pigments with the Color Index numbers CI 11725, CI 15510, CI 45370, CI 71105, or combinations thereof;
red pigments with the Color Index numbers CI 12085, CI 12120, CI 12370, CI 12420, CI 12490, CI 14700, CI 15525, CI 15580, CI 15620, CI 15630, CI 15800, CI 15850, CI15865, CI15880, CI17200, CI26100, CI45380, CI45410, CI58000, CI73360, CI 73915, CI 75470, or combinations thereof; or
combinations thereof.

10. The agent according to claim 1, wherein the at least one pigment, based on the total weight of the composition, is present in a total amount ranging from 0.01 to 10.0 wt. %.

11. The agent according to claim 1,
wherein the at least one amino-functionalized silicone polymer comprises at least one secondary amino group.

12. The agent according to claim 1, wherein the at least one amino-functionalized silicone polymer comprises at least one structural unit of the formula (Si-amino), 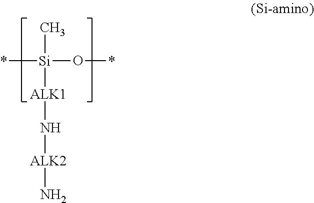
where
ALK1 and ALK2 represent, independently of one another, a linear or branched divalent C₁-C₂₀ alkylene group.

13. The agent according to claim 1, wherein the at least one amino-functionalized silicone polymer comprises structural units of the formula (Si-I) and the formula (Si-II)

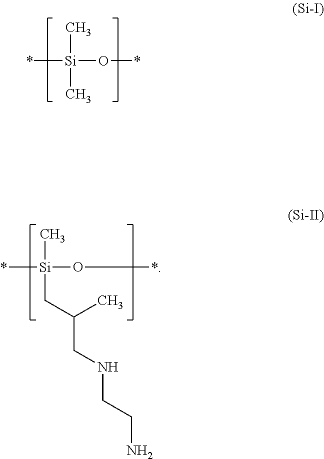

14. The agent according to claim 1, wherein the at least one amino-functionalized silicone polymer, based on the total weight of the agent, is present in an amount ranging from 0.1 to 8.0 wt %.

15. The agent according to claim 1,
further comprising at least one addition product of C₁-C₆ alkylene oxide(s) to the esters of C₁₂-C₃₀ fatty acids and aromatic C₁-C₁₂ alcohols.

16. The agent according to claim 15,
wherein the at least one addition product of C₁-C₆ alkylene oxide(s) to an ester occurred by esterification of a C₁₂-C₂₄ fatty acid with a C₁-C₁₂ aromatic alcohol selected from the group consisting of benzyl alcohol, phenol, 2-phenylethyl alcohol, or 2-phenoxyethanol.

17. The agent according to claim 15, wherein the at least one addition product, based on the total weight of the agent, is present in an amount ranging from 0.1 to 20.0 wt. %.

18. A method for dyeing keratin material, wherein the method comprises:
applying the agent of claim 1 to the keratin fibers, and
optionally rinsing out the agent after an exposure time ranging from 30 seconds to 45 minutes.
